(19) United States
(12) Patent Application Publication
(10) Pub. No.: US 2002/0106735 A1
(43) Pub. Date: Aug. 8, 2002

(54) Novel Bcl-2 related proline rich protein (BPR)

(76) Inventors: Andreas Scorilas, Athens (GR); Eleftherios Diamandis, Toronto (CA)

Correspondence Address: MERCHANT & GOULD PC, P.O. BOX 2903, MINNEAPOLIS, MN, 55402-0903, US

(21) Appl. No.: 09/953,342

(22) Filed: Sep. 14, 2001

Related U.S. Application Data
(60) Provisional application No. 60/233,026, filed on Sep. 15, 2000.

(51) Int. Cl. C12P 21/02; C07K 14/435; C12N 5/06; C07H 21/04

(52) U.S. Cl. 435/69.1; 435/325; 435/320.1; 530/350; 536/23.5

(57) ABSTRACT

The invention relates to nucleic acid molecules, proteins encoded by such nucleic acid molecules; and use of the proteins and nucleic acid molecules.

### FIELD OF THE INVENTION

The invention relates to nucleic acid molecules, proteins encoded by such nucleic acid molecules; and use of the proteins and nucleic acid molecules

### BACKGROUND OF THE INVENTION

Programmed cell death and apoptosis are highly regulated physiological processes (1, 2). Dysregulaton of apoptosis results in several diseases including cancer, loss of tissue due to ischemia and AIDS (3). Apoptotic signals induced by a number of stimuli converge into a common pathway and are executed by a family of proteins known as caspases (4, 5). Apoptotic events are regulated by a number of proteins that exert either a positive (pro-apoptotic) or a negative (anti-apoptotic) effect on programmed cell death. Proteins participating in these events include members of the Bcl-2 family (6). Members of the Bcl-2 family are characterized by the presence of at least one of the BH1, BH2, BH3 or BH4 domains (7). The BH1 and BH2 domains are present in all anti-apoptotic proteins, while the BH3 domain is present in the pro-apoptotic members of the family. However, BH3 domains have been identified in some anti-apoptotic proteins such as Bcl-2 and Bcl-X_{L} (8). The BH4 domain appears to be present in the N-terminal domain of the anti-apoptotic Bcl-2, Bcl-X_{L} and Bcl-w proteins and its function is not clear as yet. The levels of the various members of the Bcl-2 family have been shown to determine response to chemotherapy in testicular, breast and other tumors. In particular, decreased levels of Bcl-2 together with increased levels of Bax result in hypersensitivity to apoptosis (9-12).

In addition to the Bcl-2 family members, a number of Bcl-2-binding proteins have also been identified. These proteins have no significant homology with Bcl-2 or other Bcl-2 family proteins, which can form homo- and heterodimers (13-16). Although the exact mechanism by which they exert their function is not known, cellular redistribution and binding to the Bcl-2 family members appear to be critical for anti-apoptotic activity.

Proline-rich domains have been identified in a number of diverse proteins such as epidermal growth factors, phosphatidylinositol 3-kinase and, more recently, the small GTPase RRAS protein and members of the RRAS superfamily such as the TC21 protein (17-19). Proline-rich motifs are characterized by the presence of the consensus PXXP tetrapeptide, found in all proline-rich proteins identified to date (20). The hallmark of the proline-rich domain function is its interaction with SH3 domains. Interactions between SH3 domains of several tyrosine kinase oncoproteins with the proline-rich sites of their ligands mediates their oncogenic potential (17, 18, 20). Recently, it has been shown that the proline rich SH3-binding site is required for integrin activating function and the ability of RRAS to control cell adhesion (19).

### SUMMARY OF THE INVENTION

The present inventors have identified and characterized a gene encoding a novel member of the Bcl-2 family of apoptosis regulating proteins. In particular the inventors have identified, cloned, physically mapped and determined the expression pattern of BPR, a novel gene that encodes a Bcl-2 related Proline Rich protein. Proline-rich sites have been shown to interact with Src homology region 3 (SH3) domains of several tyrosine kinases, mediating their oncogenic potential. The new gene maps to chromosome 19q13.3 and is located between the IRF3 and PRMT1/HRMT1L2 genes, close to the RRAS gene. BPR is composed of seven coding exons and six intervening introns, spanning a genomic area of 8.8 kb. All of the exon/intron splice sites conform to the consensus sequence for eukaryotic splice sites. The BPR protein is composed of 334 amino acids, with a calculated molecular mass of 36.8 kDa and isoelectric point of 9.45. The BPR protein contains one BH2 homology domain, one proline-rich region similar to the TC21 protein and five consensus PXXP tetrapeptide sequences. BPR is expressed mainly in breast, thymus, prostate, fetal liver, colon, placenta, pancreas, small intestine, spinal cord, kidney and bone marrow and to a lesser extent in many other tissues. One splice variant of BPR was identified which is primarily expressed in skeletal muscle. BPR variants are expressed in normal breast and testis and that their expression is highly variable in breast and testicular cancer tissues. Given these findings, this gene is involved in the pathogenesis and/or progression of many cancers, and may find applicability as a novel cancer biomarker.

The BPR protein described herein is referred to as “BPR Protein”. The gene encoding the protein is referred to as “bpr”.

Broadly stated the present invention relates to an isolated nucleic acid molecule of at least 30 nucleotides which hybridizes to SEQ. ID. NO. 1, or the complement of SEQ ID NO. 1, under stringent hybridization conditions

The invention also contemplates a nucleic acid molecule comprising a sequence encoding a truncation of a BPR Protein, an analog, or a homolog of a BPR Protein or a truncation thereof. (BPR Protein and truncations, analogs and homologs of BPR Protein are also collectively referred to herein as “BPR Related Proteins”).

The nucleic acid molecules of the invention may be inserted into an appropriate expression vector, i.e. a vector that contains the necessary elements for the transcription and translation of the inserted coding sequence. Accordingly, recombinant expression vectors adapted for transformation of a host cell may be constructed which comprise a nucleic acid molecule of the invention and one or more transcription and translation elements linked to the nucleic acid molecule.

The recombinant expression vector can be used to prepare transformed host cells expressing BPR Related Proteins. Therefore, the invention further provides host cells containing a recombinant molecule of the invention. The invention also contemplates transgenic non-human mammals whose germ cells and somatic cells contain a recombinant molecule comprising a nucleic acid molecule of the invention, in particular one which encodes an analog of the BPR Protein, or a truncation of the BPR Protein.

The invention further provides a method for preparing BPR Related Proteins utilizing the purified and isolated nucleic acid molecules of the invention. In an embodiment a method for preparing a BPR Related Protein is provided comprising (a) transferring a recombinant expression vector of the invention into a host cell; (b) selecting transformed host cells from untransformed host cells; (c) culturing a selected transformed host cell under conditions which allow expression of the BPR Related Protein; and (d) isolating the BPR Related Protein.

The invention further broadly contemplates an isolated BPR Protein comprising an amino acid sequence of SEQ.ID.NO. 24 or 25.

The BPR Related Proteins of the invention may be conjugated with other molecules, such as proteins, to prepare fusion proteins. This may be accomplished, for example, by the synthesis of N-terminal or C-terminal fusion proteins.

The invention further contemplates antibodies having specificity against an epitope of a BPR Related Protein of the invention. Antibodies may be labeled with a detectable substance and used to detect proteins of the invention in tissues and cells. Antibodies may have particular use in therapeutic applications, for example to react with tumor cells, and in conjugates and immunotoxins as target selective carriers of various agents which have antitumor effects including chemotherapeutic drugs, toxins, immunological response modifiers, enzymes, and radioisotopes.

The invention also permits the construction of nucleotide probes which are unique to the nucleic acid molecules of the invention and/or to proteins of the invention. Therefore, the invention also relates to a probe comprising a nucleic acid sequence of the invention, or a nucleic acid sequence encoding a protein of the invention, or a part thereof. The probe may be labeled, for example, with a detectable substance and it may be used to select from a mixture of nucleotide sequences a nucleic acid molecule of the invention including nucleic acid molecules coding for a protein which displays one or more of the properties of a protein of the invention. A probe may be used to mark tumors.

The invention also provides antisense nucleic acid molecules e.g. by production of a mRNA or DNA strand in the reverse orientation to a sense molecule. An antisense nucleic acid molecule may be used to suppress the growth of a BPR expressing cell.

The invention still further provides a method for identifying a substance which binds to a protein of the invention comprising reacting the protein with at least one substance which potentially can bind with the protein, under conditions which permit the formation of complexes between the substance and protein and detecting binding. Binding may be detected by assaying for complexes, for free substance, or for non-complexed protein. The invention also contemplates methods for identifying substances that bind to other intracellular proteins that interact with a BPR Related Protein. Methods can also be utilized which identify compounds which bind to BPR gene regulatory sequences (e.g. promoter sequences).

Still further the invention provides a method for evaluating a compound for its ability to modulate the biological activity of a BPR Related Protein of the invention. For example, a substance which inhibits or enhances the interaction of the protein and a substance which binds to the protein may be evaluated. In an embodiment, the method comprises providing a known concentration of a BPR Related Protein, with a substance which binds to the protein and a test compound under conditions which permit the formation of complexes between the substance and protein, and removing and/or detecting complexes.

Compounds which modulate the biological activity of a protein of the invention may also be identified using the methods of the invention by comparing the pattern and level of expression of the protein of the invention in tissues and cells, in the presence, and in the absence of the compounds.

The proteins, antibodies, nucleic acid molecules, and substances and compounds identified using the methods of the invention, and peptides of the invention may be used to modulate the biological activity of a BPR Related Protein of the invention, and they may be used in the treatment of conditions associated with a BPR Related Protein such as cancer. Accordingly, the substances and compounds may be formulated into compositions for administration to individuals suffering from such conditions.

Therefore, the present invention also relates to a composition comprising one or more of a protein, antibody, or nucleic acid molecule of the invention, or a substance or compound identified using the methods of the invention, and a pharmaceutically acceptable carrier, excipient or diluent. A method for treating or preventing a condition associated with a BPR Related Protein (e.g. cancer) is also provided comprising administering to a patient in need thereof, a BPR Related Protein of the invention, or a composition of the invention.

Another aspect of the invention is the use of a BPR Related Protein, peptides derived therefrom, or chemically produced (synthetic) peptides, or any combination of these molecules, for use in the preparation of vaccines to prevent cancer and/or to treat cancer, in particular to prevent and/or treat cancer in patients who have a BPR Related Protein. These vaccine preparations may also be used to prevent patients from having tumors prior to their occurrence.

The invention broadly contemplates vaccines for stimulating or enhancing in a subject to whom the vaccine is administered production of antibodies directed against a BPR Related Protein.

The invention also provides a method for stimulating or enhancing in a subject production of antibodies directed against a BPR Related Protein. The method comprises administering to the subject a vaccine of the invention in a dose effective for stimulating or enhancing production of the antibodies.

The invention further provides methods for treating, preventing, or delaying recurrence of cancer. The methods comprise administering to the subject a vaccine of the invention in a dose effective for treating, preventing, or delaying recurrence of cancer.

In other embodiments, the invention provides a method for identifying inhibitors of a BPR Related Protein interaction, comprising

(a) providing a reaction mixture including the BPR Related Protein and a substance that binds to the BPR Related Protein, or at least a portion of each which interact;

(b) contacting the reaction mixture with one or more test compounds;

(c) identifying compounds which inhibit the interaction of the BPR Related Protein and substance.

In certain preferred embodiments, the reaction mixture is a whole cell. In other embodiments, the reaction mixture is a cell lysate or purified protein composition. The subject method can be carried out using libraries of test compounds. Such agents can be proteins, peptides, nucleic acids, carbohydrates, small organic molecules, and natural product extract libraries, such as those isolated from animals, plants, fungus and/or microbes.

Still another aspect of the present invention provides a method of conducting a drug discovery business comprising:

(a) providing one or more assay systems for identifying agents by their ability to inhibit or potentiate the interaction of a BPR Related Protein and a substance that binds to the protein;

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows the genomic organization and partial genomic sequence of the BPR gene. Intronic sequences are not shown except for the splice junction areas. Introns are shown with lower case letters and exons with capital letters. The coding nucleotides are shown in triplets. The translated amino acids of the coding region are shown underneath by a single letter abbreviation in bold. Part of the 5′ and 3′ genomic sequences are shown with lower case letters. For full sequence, see GenBank accession # AF289220. The start and stop codons are encircled and the exon-intron junctions are in black square boxes. BH2 homology domain is highlighted in black. The possible PXXP motifs are underlined and the TC21 identical motif is doubly lined from above. The putative TATA box and polyadenylation signal are in bold.

FIG. 2 shows the alignment of the BH2 deduced amino acid sequence of BPR with members of the Bcl-2 multigene family (SEQ ID NOS. 17-23). Identical amino acids are highlighted in black and similar residues in grey.

FIG. 3 shows a plot of hydrophobicity and hydrophilicity of the BPR protein.

FIG. 4 is a schematic presentation of the different splice variants of the BPR gene. Exons are shown by boxes and introns by the connecting lines. Numbers inside boxes represent the exons lengths in base pairs. The arrowhead points to the common start codon and stars to the stop codon positions. The length of the predicted polypeptide product is indicated beside each variant in amino acids (AA). For full sequence information, see GenBank submission # AF289220. The alternative splicing and/or exon skips create a frame shift, which will lead to a premature termination of translation.

FIG. 5 shows the relative locations of the PRMT1/HRMT1L2, BPR, IRF3 and RRAS, on chromosome 19q13.3. Genes are represented by horizontal arrows denoting the direction of the coding sequence. Distances between genes are mentioned in base pairs. Figure is not drawn to scale.

FIG. 6 shows the tissue expression of the BPR gene, as determined by RT-PCR. The splice variant 1 (lower PCR band) is higly expressed in skeletal muscle. PCR was performed with primers BPR-F2 and BPR-R6 (Table 2).

FIG. 7 shows the expression of the BPR gene in the testis and breast. M, DNA molecular weight marker; ct, negative control; N_{T}, normal testis tissue; N_{B}, normal breast tissue. Note low or complete absence of expression in some of cancer tissues. For isoforms see also FIG. 4.

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

1. Nucleic Acid Molecules of the Invention

As hereinbefore mentioned, the invention provides an isolated nucleic acid molecule having a sequence encoding a BPR Related Protein. The term “isolated” refers to a nucleic acid substantially free of cellular material or culture medium when produced by recombinant DNA techniques, or chemical reactants, or other chemicals when chemically synthesized. An “isolated” nucleic acid may also be free of sequences which naturally flank the nucleic acid (i.e., sequences located at the 5′ and 3′ ends of the nucleic acid molecule) from which the nucleic acid is derived. The term “nucleic acid” is intended to include DNA and RNA and can be either double stranded or single stranded. In an embodiment, a nucleic acid molecule encodes a BPR Related Protein comprising an amino acid sequence of SEQ.ID.NO. 24 or 25, preferably a nucleic acid molecule comprising a nucleic acid sequence of one of SEQ.ID.NOs. 1 to 8.

In an embodiment, the invention provides an isolated nucleic acid molecule which comprises:

(i) a nucleic acid sequence encoding a protein having substantial sequence identity with an amino acid sequence of SEQ.ID.NO. 24 or 25;

(ii) a nucleic acid sequence encoding a protein comprising an amino acid sequence of SEQ.ID.NO. 24 or 25;

(iii) nucleic acid sequences complementary to (i);

(iv) a degenerate form of a nucleic acid sequence of (i);

(v) a nucleic acid sequence capable of hybridizing under stringent conditions to a nucleic acid sequence in (i), (ii) or (iii);

(vi) a nucleic acid sequence encoding a truncation, an analog, an allelic or species variation of a protein comprising an amino acid sequence of SEQ.ID.NO. 24 or 25; or

(vii) a fragment, or allelic or species variation of (i), (ii) or (iii).

Preferably, a purified and isolated nucleic acid molecule of the invention comprises:

(i) a nucleic acid sequence comprising the sequence of one of SEQ.ID.NOs. 1 to 8 wherein T can also be U;

(ii) nucleic acid sequences complementary to (i), preferably complementary to the full nucleic acid sequence of one of SEQ.ID.NOs. 1 to 8;

(iii) a nucleic acid capable of hybridizing under stringent conditions to a nucleic acid of (i) or (ii) and preferably having at least 18 nucleotides; or

(iv) a nucleic acid molecule differing from any of the nucleic acids of (i) to (iii) in codon sequences due to the degeneracy of the genetic code.

The invention includes nucleic acid sequences complementary to a nucleic acid encoding a BPR Protein comprising an amino acid sequence of SEQ.ID.NO. 24 or 25, preferably the nucleic acid sequences complementary to a full nucleic acid sequence of one of SEQ.ID.NOs. 1 to 8.

The invention includes nucleic acid molecules having substantial sequence identity or homology to nucleic acid sequences of the invention or encoding proteins having substantial identity or similarity to the amino acid sequence of SEQ.ID.NOs. 24 or 25. Preferably, the nucleic acids have substantial sequence identity for example at least 50%, 55%, 60%, 65%, 70%, 75%, 80%, or 85% nucleic acid identity; more preferably 90% nucleic acid identity; and most preferably at least 95%, 96%, 97%, 98%, or 99% sequence identity. “Identity” as known in the art and used herein, is a relationship between two or more amino acid sequences or two or more nucleic acid sequences, as determined by comparing the sequences. It also refers to the degree of sequence relatedness between amino acid or nucleic acid sequences, as the case may be, as determined by the match between strings of such sequences. Identity and similarity are well known terms to skilled artisans and they can be calculated by conventional methods (for example see Computational Molecular Biology, Lesk, A. M. ed., Oxford University Press, New York, 1988; Biocomputing: Informatics and Genome Projects, Smith, D. W. ed., Academic Press, New York, 1993; Computer Analysis of Sequence Data, Part I, Griffin, A. M. and Griffin, H. G. eds., Humana Press, New Jersey, 1994; Sequence Analysis in Molecular Biology, von Heinje, G. Acadmeic Press, 1987; and Sequence Analysis Primer, Gribskov, M. and Devereux, J. eds. M. Stockton Press, New York, 1991, Carillo, H. and Lipman, D., SIAM J. Applied Math. 48:1073, 1988). Methods which are designed to give the largest match between the sequences are generally preferred. Methods to determine identity and similarity are codified in publicly available computer programs including the GCG program package (Devereux J. et al., Nucleic Acids Research 12(1): 387,1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990).

Isolated nucleic acid molecules encoding a BPR Protein, and having a sequence which differs from a nucleic acid sequence of the invention due to degeneracy in the genetic code are also within the scope of the invention. Such nucleic acids encode functionally equivalent proteins (e.g. a BPR Protein) but differ in sequence from the sequence of a BPR Protein due to degeneracy in the genetic code. As one example, DNA sequence polymorphisms within the nucleotide sequence of a BPR Protein may result in silent mutations which do not affect the amino acid sequence. Variations in one or more nucleotides may exist among individuals within a population due to natural allelic variation. Any and all such nucleic acid variations are within the scope of the invention. DNA sequence polymorphisms may also occur which lead to changes in the amino acid sequence of a BPR Protein. These amino acid polymorphisms are also within the scope of the present invention.

Another aspect of the invention provides a nucleic acid molecule which hybridizes under stringent conditions, preferably high stringency conditions to a nucleic acid molecule which comprises a sequence which encodes a BPR Protein having an amino acid sequence shown in SEQ.ID.NO. 24 or 25. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0× SSC at 50° C. may be employed. The stringency may be selected based on the conditions used in the wash step. By way of example, the salt concentration in the wash step can be selected from a high stringency of about 0.2× SSC at 50° C. In addition, the temperature in the wash step can be at high stringency conditions, at about 65° C.

It will be appreciated that the invention includes nucleic acid molecules encoding a BPR Related Protein including truncations of a BPR Protein, and analogs of a BPR Protein as described herein. It will further be appreciated that variant forms of the nucleic acid molecules of the invention which arise by alternative splicing of an mRNA corresponding to a cDNA of the invention are encompassed by the invention.

An isolated nucleic acid molecule of the invention which comprises DNA can be isolated by preparing a labelled nucleic acid probe based on all or part of a nucleic acid sequence of the invention. The labeled nucleic acid probe is used to screen an appropriate DNA library (e.g. a cDNA or genomic DNA library). For example, a cDNA library can be used to isolate a cDNA encoding a BPR Related Protein by screening the library with the labeled probe using standard techniques. Alternatively, a genomic DNA library can be similarly screened to isolate a genomic clone encompassing a gene encoding a BPR Related Protein. Nucleic acids isolated by screening of a cDNA or genomic DNA library can be sequenced by standard techniques.

An isolated nucleic acid molecule of the invention which is DNA can also be isolated by selectively amplifying a nucleic acid encoding a BPR Related Protein using the polymerase chain reaction (PCR) methods and cDNA or genomic DNA. It is possible to design synthetic oligonucleotide primers from the nucleotide sequence of the invention for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the invention which is RNA can be isolated by cloning a cDNA encoding a BPR Related Protein into an appropriate vector which allows for transcription of the cDNA to produce an RNA molecule which encodes a BPR Related Protein. For example, a cDNA can be cloned downstream of a bacteriophage promoter, (e.g. a T7 promoter) in a vector, cDNA can be transcribed in vitro with T7 polymerase, and the resultant RNA can be isolated by conventional techniques.

Nucleic acid molecules of the invention may be chemically synthesized using standard techniques. Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular nucleic acid molecule encodes a BPR Related Protein can be accomplished by expressing the cDNA in an appropriate host cell by standard techniques, and testing the expressed protein in the methods described herein. A cDNA encoding a BPR Related Protein can be sequenced by standard techniques, such as dideoxynucleotide chain termination or Maxam-Gilbert chemical sequencing, to determine the nucleic acid sequence and the predicted amino acid sequence of the encoded protein.

The initiation codon and untranslated sequences of a BPR Related Protein may be determined using computer software designed for the purpose, such as PC/Gene (IntelliGenetics Inc., Calif.). The intron-exon structure and the transcription regulatory sequences of a gene encoding a BPR Related Protein may be confirmed by using a nucleic acid molecule of the invention encoding a BPR Related Protein to probe a genomic DNA clone library. Regulatory elements can be identified using standard techniques. The function of the elements can be confirmed by using these elements to express a reporter gene such as the lacZ gene which is operatively linked to the elements. These constructs may be introduced into cultured cells using conventional procedures or into non-human transgenic animal models. In addition to identifying regulatory elements in DNA, such constructs may also be used to identify nuclear proteins interacting with the elements, using techniques known in the art.

In a particular embodiment of the invention, the nucleic acid molecules isolated using the methods described herein are mutant bpr gene alleles. The mutant alleles may be isolated from individuals either known or proposed to have a genotype which contributes to the symptoms of a disorder (e.g. cancer). Mutant alleles and mutant allele products may be used in therapeutic and diagnostic methods described herein. For example, a cDNA of a mutant bpr gene may be isolated using PCR as described herein, and the DNA sequence of the mutant allele may be compared to the normal allele to ascertain the mutation(s) responsible for the loss or alteration of function of the mutant gene product. A genomic library can also be constructed using DNA from an individual suspected of or known to carry a mutant allele, or a cDNA library can be constructed using RNA from tissue known, or suspected to express the mutant allele. A nucleic acid encoding a normal bpr gene or any suitable fragment thereof, may then be labeled and used as a probe to identify the corresponding mutant allele in such libraries. Clones containing mutant sequences can be purified and subjected to sequence analysis. In addition, an expression library can be constructed using cDNA from RNA isolated from a tissue of an individual known or suspected to express a mutant bpr allele. Gene products made by the putatively mutant tissue may be expressed and screened, for example using antibodies specific for a BPR Related Protein as described herein. Library clones identified using the antibodies can be purified and subjected to sequence analysis.

The sequence of a nucleic acid molecule of the invention, or a fragment of the molecule, may be inverted relative to its normal presentation for transcription to produce an antisense nucleic acid molecule. An antisense nucleic acid molecule may be constructed using chemical synthesis and enzymatic ligation reactions using procedures known in the art.

2. Proteins of the Invention

An amino acid sequence of a BPR Protein comprises a sequence as shown in SEQ.ID.NO. 24 or 25. The protein is expressed mainly in breast, thymus, prostate, fetal liver, colon, placenta, pancreas, small intestine, spinal cord, kidney, and bone marrow. A splice variant of the protein (SEQ ID NO 25) is primarily expressed in skeletal muscle.

In addition to proteins comprising an amino acid sequence as shown in SEQ.ID.NO. 24 or 25, the proteins of the present invention include truncations of a BPR Protein, analogs of a BPR Protein, and proteins having sequence identity or similarity to a BPR Protein, and truncations thereof as described herein (i.e. BPR Related Proteins). Truncated proteins may comprise peptides of between 3 and 70 amino acid residues, ranging in size from a tripeptide to a 70 mer polypeptide (e.g. WIQAHGGW and EGILAVSP—SEQ ID Nos. 26 and 27).

The truncated proteins may have an amino group (—NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated proteins may have a carboxyl group, an amido group, a T-butyloxycarbonyl group, or a macromolecule including but not limited to lipid-fatty acid conjugates, polyethylene glycol, or carbohydrates at the carboxy terminal end.

The proteins of the invention may also include analogs of a BPR Protein, and/or truncations thereof as described herein, which may include, but are not limited to a BPR Protein, containing one or more amino acid substitutions, insertions, and/or deletions. Amino acid substitutions may be of a conserved or non-conserved nature. Conserved amino acid substitutions involve replacing one or more amino acids of a BPR Protein amino acid sequence with amino acids of similar charge, size, and/or hydrophobicity characteristics. When only conserved substitutions are made the resulting analog is preferably functionally equivalent to a BPR Protein. Non-conserved substitutions involve replacing one or more amino acids of the BPR Protein amino acid sequence with one or more amino acids which possess dissimilar charge, size, and/or hydrophobicity characteristics.

One or more amino acid insertions may be introduced into a BPR Protein. Amino acid insertions may consist of single amino acid residues or sequential amino acids ranging from 2 to 15 amino acids in length.

Deletions may consist of the removal of one or more amino acids, or discrete portions from a BPR Protein sequence. The deleted amino acids may or may not be contiguous. The lower limit length of the resulting analog with a deletion mutation is about 10 amino acids, preferably 20 to 40 amino acids.

The proteins of the invention include proteins with sequence identity or similarity to a BPR Protein and/or truncations thereof as described herein. Such BPR Proteins include proteins whose amino acid sequences are comprised of the amino acid sequences of BPR Protein regions from other species that hybridize under selected hybridization conditions (see discussion of stringent hybridization conditions herein) with a probe used to obtain a BPR Protein. These proteins will generally have the same regions which are characteristic of a BPR Protein. Preferably a protein will have substantial sequence identity for example, about 65%, 70%, 75%, 80%, or 85% identity, preferably 90% identity, more preferably at least 95%, 96%, 97%, 98%, or 99% identity, and most preferably 98% identity with an amino acid sequence shown in in SEQ.ID.NO. 24 or 25. A percent amino acid sequence homology, similarity or identity is calculated as the percentage of aligned amino acids that match the reference sequence using known methods as described herein.

The invention also contemplates isoforms of the proteins of the invention. An isoform contains the same number and kinds of amino acids as a protein of the invention, but the isoform has a different molecular structure. Isoforms contemplated by the present invention preferably have the same properties as a protein of the invention as described herein.

The present invention also includes BPR Related Proteins conjugated with a selected protein, or a marker protein (see below) to produce fusion proteins. Additionally, immunogenic portions of a BPR Protein and a BPR Protein Related Protein are within the scope of the invention.

A BPR Related Protein of the invention may be prepared using recombinant DNA methods. Accordingly, the nucleic acid molecules of the present invention having a sequence which encodes a BPR Related Protein of the invention may be incorporated in a known manner into an appropriate expression vector which ensures good expression of the protein. Possible expression vectors include but are not limited to cosmids, plasmids, or modified viruses (e.g. replication defective retroviruses, adenoviruses and adeno-associated viruses), so long as the vector is compatible with the host cell used.

The invention therefore contemplates a recombinant expression vector of the invention containing a nucleic acid molecule of the invention, and the necessary regulatory sequences for the transcription and translation of the inserted protein-sequence. Suitable regulatory sequences may be derived from a variety of sources, including bacterial, fungal, viral, mammalian, or insect genes [For example, see the regulatory sequences described in Goeddel, Gene Expression Technology: Methods in Enzymology 185, Academic Press, San Diego, Calif. (1990)]. Selection of appropriate regulatory sequences is dependent on the host cell chosen as discussed below, and may be readily accomplished by one of ordinary skill in the art. The necessary regulatory sequences may be supplied by the native BPR Protein and/or its flanking regions.

The invention further provides a recombinant expression vector comprising a DNA nucleic acid molecule of the invention cloned into the expression vector in an antisense orientation. That is, the DNA molecule is linked to a regulatory sequence in a manner which allows for expression, by transcription of the DNA molecule, of an RNA molecule which is antisense to the nucleic acid sequence of a protein of the invention or a fragment thereof. Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA.

The recombinant expression vectors of the invention may also contain a marker gene which facilitates the selection of host cells transformed or transfected with a recombinant molecule of the invention. Examples of marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

The recombinant expression vectors may be introduced into host cells to produce a transformant host cell. “Transformant host cells” include host cells which have been transformed or transfected with a recombinant expression vector of the invention. The terms “transformed with”, “transfected with”, “transformation” and “transfection” encompass the introduction of a nucleic acid (e.g. a vector) into a cell by one of many standard techniques. Prokaryotic cells can be transformed with a nucleic acid by, for example, electroporation or calcium-chloride mediated transformation. A nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells, or mammalian cells. Other suitable host cells can be found in Goeddel, Gene Expression Technology: Methods in Enzymology 185, Academic Press, San Diego, Calif. (1991).

A host cell may also be chosen which modulates the expression of an inserted nucleic acid sequence, or modifies (e.g. glycosylation or phosphorylation) and processes (e.g. cleaves) the protein in a desired fashion. Host systems or cell lines may be selected which have specific and characteristic mechanisms for post-translational processing and modification of proteins. For example, eukaryotic host cells including CHO, VERO, BHK, HeLA, COS, MDCK, 293, 3T3, and WI38 may be used. For long-term high-yield stable expression of the protein, cell lines and host systems which stably express the gene product may be engineered.

Host cells and in particular cell lines produced using the methods described herein may be particularly useful in screening and evaluating compounds that modulate the activity of a BPR Related Protein.

The proteins of the invention may also be expressed in non-human transgenic animals including but not limited to mice, rats, rabbits, guinea pigs, micro-pigs, goats, sheep, pigs, non-human primates (e.g. baboons, monkeys, and chimpanzees) [see Hammer et al. (Nature 315:680-683, 1985), Palmiter et al. (Science 222:809-814, 1983), Brinster et al. (Proc Natl. Acad. Sci USA 82:44384442, 1985), Palmiter and Brinster (Cell. 41:343-345, 1985) and U.S. Pat. No. 4,736,866)]. Procedures known in the art may be used to introduce a nucleic acid molecule of the invention encoding a BPR Related Protein into animals to produce the founder lines of transgenic animals. Such procedures include pronuclear microinjection, retrovirus mediated gene transfer into germ lines, gene targeting in embryonic stem cells, electroporation of embryos, and sperm-mediated gene transfer.

The present invention contemplates a transgenic animal that carries the BPR gene in all their cells, and animals which carry the transgene in some but not all their cells. The transgene may be integrated as a single transgene or in concatamers. The transgene may be selectively introduced into and activated in specific cell types (See for example, Lasko et al, 1992 Proc. Natl. Acad. Sci. USA 89: 6236). The transgene may be integrated into the chromosomal site of the endogenous gene by gene targeting. The transgene may be selectively introduced into a particular cell type inactivating the endogenous gene in that cell type (See Gu et al Science 265: 103-106).

The expression of a recombinant BPR Related Protein in a transgenic animal may be assayed using standard techniques. Initial screening may be conducted by Southern Blot analysis, or PCR methods to analyze whether the transgene has been integrated. The level of mRNA expression in the tissues of transgenic animals may also be assessed using techniques including Northern blot analysis of tissue samples, in situ hybridization, and RT-PCR. Tissue may also be evaluated immunocytochemically using antibodies against BPR Protein.

Proteins of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

N-terminal or C-terminal fusion proteins comprising a BPR Related Protein of the invention conjugated with other molecules, such as proteins, may be prepared by fusing, through recombinant techniques, the N-terminal or C-terminal of a BPR Related Protein, and the sequence of a selected protein or marker protein with a desired biological function. The resultant fusion proteins contain BPR Protein fused to the selected protein or marker protein as described herein. Examples of proteins which may be used to prepare fusion proteins include immunoglobulins, glutathione-S-transferase (GST), hemagglutinin (HA), and truncated myc.

3. Antibodies

BPR Related Proteins of the invention can be used to prepare antibodies specific for the proteins. Antibodies can be prepared which bind a distinct epitope in an unconserved region of the protein. An unconserved region of the protein is one that does not have substantial sequence homology to other proteins. A region from a conserved region such as a well-characterized domain can also be used to prepare an antibody to a conserved region of a BPR Related Protein. Antibodies having specificity for a BPR Related Protein may also be raised from fusion proteins created by expressing fusion proteins in bacteria as described herein.

The invention can employ intact monoclonal or polyclonal antibodies, and immunologically active fragments (e.g. a Fab, (Fab)₂ fragment, or Fab expression library fragments and epitope-binding fragments thereof), an antibody heavy chain, and antibody light chain, a genetically engineered single chain Fv molecule (Ladner et al, U.S. Pat. No. 4,946,778), humanized antibodies, or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

4. Applications of the Nucleic Acid Molecules, BPR Related Proteins, and Antibodies of the Invention

The nucleic acid molecules, BPR Related Proteins, and antibodies of the invention may be used in the prognostic and diagnostic evaluation of conditions associated with a BPR Related Protein such as cancer, and the identification of subjects with a predisposition to such conditions (Section 4.1.1 and 4.1.2).

In an embodiment of the invention, a method is provided for detecting the expression of a BPR Related Protein in a patient comprising:

(a) taking a sample derived from a patient; and

(b) detecting in the sample a nucleic acid sequence encoding the BPR Related Protein or a protein product encoded by a BPR nucleic acid sequence.

In a particular embodiment of the invention, the nucleic acid molecules, BPR Related Proteins, and antibodies of the invention may be used in the diagnosis and staging of cancer.

Methods for detecting nucleic acid molecules and BPR Related Proteins of the invention, can be used to monitor conditions such as cancer by detecting BPR Related Proteins and nucleic acid molecules encoding BPR Related Proteins. The applications of the present invention also include methods for the identification of compounds that modulate the biological activity of BPR or BPR Related Proteins (Section 4.2). The compounds, antibodies etc. may be used for the treatment of conditions associated with a BPR Related Protein such as cancer (Section 4.3). It would also be apparent to one skilled in the art that the methods described herein may be used to study the developmental expression of BPR Related Proteins and, accordingly, will provide further insight into the role of BPR Related Proteins

4.1 Diagnostic Methods

A variety of methods can be employed for the diagnostic and prognostic evaluation of conditions associated with a BPR Related Protein such as cancer, and the identification of subjects with a predisposition to such conditions. Such methods may, for example, utilize nucleic acid molecules of the invention, and fragments thereof, and antibodies directed against BPR Related Proteins, including peptide fragments. In particular, the nucleic acids and antibodies may be used, for example, for: (1) the detection of the presence of BPR mutations, or the detection of either over- or under-expression of BPR mRNA relative to a non-disorder state or the qualitative or quantitative detection of alternatively spliced forms of BPR transcripts which may correlate with certain conditions or susceptibility toward such conditions; and (2) the detection of either an over- or an under-abundance of BPR Related Proteins relative to a non-disorder state or the presence of a modified (e.g., less than full length) BPR Protein which correlates with a disorder state, or a progression toward a disorder state.

The methods described herein may be performed by utilizing pre-packaged diagnostic kits comprising at least one specific BPR nucleic acid or antibody described herein, which may be conveniently used, e.g., in clinical settings, to screen and diagnose patients and to screen and identify those individuals exhibiting a predisposition to developing a disorder.

Nucleic acid-based detection techniques are described, below, in Section 4.1.1. Peptide detection techniques are described, below, in Section 4.1.2. The samples that may be analyzed using the methods of the invention include those which are known or suspected to express BPR or contain BPR Related Proteins. The samples may be derived from a patient or a cell culture, and include but are not limited to biological fluids, tissue extracts, freshly harvested cells, and lysates of cells which have been incubated in cell cultures.

Oligonucleotides or longer fragments derived from any of the nucleic acid molecules of the invention may be used as targets in a microarray. The microarray can be used to simultaneously monitor the expression levels of large numbers of genes and to identify genetic variants, mutations, and polymorphisms. The information from the microarray may be used to determine gene function, to understand the genetic basis of a disorder, to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

4.1.1 Methods for Detecting Nucleic Acid Molecules of the Invention

The nucleic acid molecules of the invention allow those skilled in the art to construct 25 nucleotide probes for use in the detection of nucleic acid sequences of the invention in samples.

Suitable probes include nucleic acid molecules based on nucleic acid sequences encoding at least 5 sequential amino acids from regions of the BPR Protein, preferably they comprise 15 to 30 nucleotides. A nucleotide probe may be labeled with a detectable substance such as a radioactive label which provides for an adequate signal and has sufficient half-life such as ³²P, ³H, ¹⁴C or the like. Other detectable substances which may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect genes, preferably in human cells, that encode BPR Related Proteins. The nucleotide probes may also be useful in the diagnosis of cancer; in monitoring the progression of cancer; or monitoring a therapeutic treatment.

The probe may be used in hybridization techniques to detect genes that encode BPR Related Proteins. The technique generally involves contacting and incubating nucleic acids (e.g. recombinant DNA molecules, cloned genes) obtained from a sample from a patient or other cellular source with a probe of the present invention under conditions favorable for the specific annealing of the probes to complementary sequences in the nucleic acids. After incubation, the non-annealed nucleic acids are removed, and the presence of nucleic acids that have hybridized to the probe if any are detected.

The detection of nucleic acid molecules of the invention may involve the amplification of specific gene sequences using an amplification method such as PCR, followed by the analysis of the amplified molecules using techniques known to those skilled in the art. Suitable primers can be routinely designed by one of skill in the art.

Genomic DNA may be used in hybridization or amplification assays of biological samples to detect abnormalities involving BPR structure, including point mutations, insertions, deletions, and chromosomal rearrangements. For example, direct sequencing, single stranded conformational polymorphism analyses, heteroduplex analysis, denaturing gradient gel electrophoresis, chemical mismatch cleavage, and oligonucleotide hybridization may be utilized.

Genotyping techniques known to one skilled in the art can be used to type polymorphisms that are in close proximity to the mutations in a bpr gene. The polymorphisms may be used to identify individuals in families that are likely to carry mutations. If a polymorphism exhibits linkage disequalibrium with mutations in a bpr gene, it can also be used to screen for individuals in the general population likely to carry mutations. Polymorphisms which may be used include restriction fragment length polymorphisms (RFLPs), single-base polymorphisms, and simple sequence repeat polymorphisms (SSLPs).

A probe of the invention may be used to directly identify RFLPs. A probe or primer of the invention can additionally be used to isolate genomic clones such as YACs, BACs, PACs, cosmids, phage or plasmids. The DNA in the clones can be screened for SSLPs using hybridization or sequencing procedures.

Hybridization and amplification techniques described herein may be used to assay qualitative and quantitative aspects of bpr expression. For example, RNA may be isolated from a cell type or tissue known to express bpr and tested utilizing the hybridization (e.g. standard Northern analyses) or PCR techniques referred to herein. The techniques may be used to detect differences in transcript size which may be due to normal or abnormal alternative splicing. The techniques may be used to detect quantitative differences between levels of full length and/or alternatively splice transcripts detected in normal individuals relative to those individuals exhibiting symptoms of a disorder involving a BPR Related Protein.

The primers and probes may be used in the above described methods in situ i.e directly on tissue sections (fixed and/or frozen) of patient tissue obtained from biopsies or resections.

4.1.2 Methods for Detecting BPR Related Proteins

Antibodies specifically reactive with a BPR Related Protein, or derivatives, such as enzyme conjugates or labeled derivatives, may be used to detect BPR Related Proteins in various samples (e.g. biological materials). They may be used as diagnostic or prognostic reagents and they may be used to detect abnormalities in the level of BPR Related Protein expression, or abnormalities in the structure, and/or temporal, tissue, cellular, or subcellular location of a BPR Related Protein. Antibodies may also be used to screen potentially therapeutic compounds in vitro to determine their effects on conditions including cancer. In vitro immunoassays may also be used to assess or monitor the efficacy of particular therapies. The antibodies of the invention may also be used in vitro to determine the level of bpr expression in cells genetically engineered to produce a BPR Related Protein.

The antibodies may be used in any known immunoassays which rely on the binding interaction between an antigenic determinant of a BPR Related Protein and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify BPR Related Proteins in a sample in order to determine its role in particular cellular events or pathological states, and to diagnose and treat such pathological states.

In particular, the antibodies of the invention may be used in immuno-histochemical analyses, for example, at the cellular and sub-subcellular level, to detect a BPR Related Protein, to localize it to particular cells and tissues, and to specific subcellular locations, and to quantitate the level of expression.

Cytochemical techniques known in the art for localizing antigens using light and electron microscopy may be used to detect a BPR Related Protein. Generally, an antibody of the invention may be labeled with a detectable substance and a BPR Related Protein may be localised in tissues and cells based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

The antibody or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against a BPR Related Protein. By way of example, if the antibody having specificity against a BPR Related Protein is a rabbit IgG antibody, the second antibody may be goat anti-rabbit gamma-globulin labeled with a detectable substance as described herein.

Where a radioactive label is used as a detectable substance, a BPR Related Protein may be localized by radioautography. The results of radioautography may be quantitated by determining the density of particles in the radioautographs by various optical methods, or by counting the grains.

In an embodiment, the invention contemplates a method for monitoring the progression of a disorder involving a BPR Related Protein (e.g. cancer) in an individual, comprising:

(a) contacting an amount of an antibody which binds to a BPR Related Protein, with a sample from the individual so as to form a binary complex comprising the antibody and BPR Related Protein in the sample;

(b) determining or detecting the presence or amount of complex formation in the sample;

(c) repeating steps (a) and (b) at a point later in time; and

(d) comparing the result of step (b) with the result of step (c), wherein a difference in the amount of complex formation is indicative of the progression of the disorder in said individual.

The amount of complexes may also be compared to a value representative of the amount of the complexes from an individual not at risk of, or afflicted with, the disorder (e.g. cancer).

4.2 Methods for Identifying or Evaluating Substances/Compounds

The methods described herein are designed to identify substances that modulate the biological activity of a BPR Related Protein including substances that bind to BPR Related Proteins, or bind to other proteins that interact with a BPR Related Protein, to compounds that interfere with, or enhance the interaction of a BPR Related Protein and substances that bind to the BPR Related Protein or other proteins that interact with a BPR Related Protein. Methods are also utilized that identify compounds that bind to BPR regulatory sequences.

The substances and compounds identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The substance or compound may be an endogenous physiological compound or it may be a natural or synthetic compound.

Substances which modulate a BPR Related Protein can be identified based on their ability to bind to a BPR Related Protein. Therefore, the invention also provides methods for identifying substances which bind to a BPR Related Protein. Substances identified using the methods of the invention may be isolated, cloned and sequenced using conventional techniques. A substance that associates with a polypeptide of the invention may be an agonist or antagonist of the biological or immunological activity of a polypeptide of the invention.

The term “agonist” refers to a molecule that increases the amount of, or prolongs the duration of, the activity of the protein. The term “antagonist” refers to a molecule which decreases the biological or immunological activity of the protein. Agonists and antagonists may include proteins, nucleic acids, carbohydrates, or any other molecules that associate with a protein of the invention.

Substances which can bind with a BPR Related Protein may be identified by reacting a BPR Related Protein with a test substance which potentially binds to a BPR Related Protein, under conditions which permit the formation of substance-BPR Related Protein complexes, and removing and/or detecting the complexes. The complexes can be detected by assaying for substance-BPR Related Protein complexes, for free substance, or for non-complexed BPR Related Protein. Conditions which permit the formation of substance-BPR Related Protein complexes may be selected having regard to factors such as the nature and amounts of the substance and the protein.

The substance-protein complex, free substance or non-complexed proteins may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against BPR Related Protein or the substance, or labeled BPR Related Protein, or a labeled substance maybe utilized. The antibodies, proteins, or substances may be labeled with a detectable substance as described above.

A BPR Related Protein, or the substance used in the method of the invention may be insolubilized. For example, a BPR Related Protein, or substance may be bound to a suitable carrier such as agarose, cellulose, dextran, Sephadex, Sepharose, carboxymethyl cellulose, polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized protein or substance may be prepared by reacting the material with a suitable insoluble carrier using known chemical or physical methods, for example, cyanogen bromide coupling.

The invention also contemplates a method for evaluating a compound for its ability to modulate the biological activity of a BPR Related Protein of the invention, by assaying for an agonist or antagonist (i.e. enhancer or inhibitor) of the binding of a BPR Related Protein with a substance which binds with a BPR Related Protein. Examples of such substances include SH3 containing proteins such as c-Crk, Fyn, Alb tyrosine kinase, phosphatidyklinositol 3-kinase, hematopoietic cell kinase HCK, c-src tyrosine kinase, GRB2, phospholipase C, Src kinase, and p56-lck tyrosine kinase. The basic method for evaluating if a compound is an agonist or antagonist of the binding of a BPR Related Protein and a substance that binds to the protein, is to prepare a reaction mixture containing the BPR Related Protein and the substance under conditions which permit the formation of substance-BPR Related Protein complexes, in the presence of a test compound. The test compound may be initially added to the mixture, or may be added subsequent to the addition of the BPR Related Protein and substance. Control reaction mixtures without the test compound or with a placebo are also prepared. The formation of complexes is detected and the formation of complexes in the control reaction but not in the reaction mixture indicates that the test compound interferes with the interaction of the BPR Related Protein and substance. The reactions may be carried out in the liquid phase or the BPR Related Protein, substance, or test compound may be immobilized as described herein. The ability of a compound to modulate the biological activity of a BPR Related Protein of the invention may be tested by determining the biological effects on cells.

It will be understood that the agonists and antagonists i.e. inhibitors and enhancers, that can be assayed using the methods of the invention may act on one or more of the binding sites on the protein or substance including agonist binding sites, competitive antagonist binding sites, non-competitive antagonist binding sites or allosteric sites.

The invention also makes it possible to screen for antagonists that inhibit the effects of an agonist of the interaction of a BPR Related Protein with a substance that is capable of binding to the BPR Related Protein. Thus, the invention may be used to assay for a compound that competes for the same binding site of a BPR Related Protein.

The invention also contemplates methods for identifying compounds that bind to proteins that interact with a BPR Related Protein. Protein-protein interactions may be identified using conventional methods such as co-immunoprecipitation, crosslinking and co-purification through gradients or chromatographic columns. Methods may also be employed that result in the simultaneous identification of genes which encode proteins interacting with a BPR Related Protein. These methods include probing expression libraries with labeled BPR Related Protein.

Two-hybrid systems may also be used to detect protein interactions in vivo. Generally, plasmids are constructed that encode two hybrid proteins. A first hybrid protein consists of the DNA-binding domain of a transcription activator protein fused to a BPR Related Protein, and the second hybrid protein consists of the transcription activator protein's activator domain fused to an unknown protein encoded by a cDNA which has been recombined into the plasmid as part of a cDNA library. The plasmids are transformed into a strain of yeast (e.g. S. cerevisiae) that contains a reporter gene (e.g. lacZ, luciferase, alkaline phosphatase, horseradish peroxidase) whose regulatory region contains the transcription activator's binding site. The hybrid proteins alone cannot activate the transcription of the reporter gene. However, interaction of the two hybrid proteins reconstitutes the functional activator protein and results in expression of the reporter gene, which is detected by an assay for the reporter gene product.

It will be appreciated that fusion proteins may be used in the above-described methods. In particular, BPR Related Proteins fused to a glutathione-S-transferase may be used in the methods.

The reagents suitable for applying the methods of the invention to evaluate compounds that modulate a BPR Related Protein may be packaged into convenient kits providing the necessary materials packaged into suitable containers. The kits may also include suitable supports useful in performing the methods of the invention.

4.3 Compositions and Treatments

The proteins of the invention, substances or compounds identified by the methods described herein, antibodies, and antisense nucleic acid molecules of the invention may be used for modulating the biological activity of a BPR Related Protein, and they may be used in the treatment of conditions associated with a BPR Related Protein such as cancer, in particular testicular, breast, and ovarian cancer.

The substances, antibodies, and compounds may be formulated into pharmaceutical compositions for administration to subjects in a biologically compatible form suitable for administration in vivo. By “biologically compatible form suitable for administration in vivo” is meant a form of the active substance to be administered in which any toxic effects are outweighed by the therapeutic effects. The active substances may be administered to living organisms including humans, and animals. Administration of a therapeutically active amount of a pharmaceutical composition of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment (e.g. chemotherapy or radiotherapy). For example, the compositions may be used in combination with anti-proliferative agents, antimicrobial agents, immunostimulatory agents, or anti-inflammatories. The compositions of the invention may be administered concurrently, separately, or sequentially with other therapeutic agents or therapies.

Vectors derived from retroviruses, adenovirus, herpes or vaccinia viruses, or from various bacterial plasmids, may be used to deliver nucleic acid molecules to a targeted organ, tissue, or cell population. Methods well known to those skilled in the art may be used to construct recombinant vectors which will express antisense nucleic acid molecules of the invention. (See, for example, the techniques described in Sambrook et al (supra) and Ausubel et al (supra)).

The nucleic acid molecules comprising full length cDNA sequences and/or their regulatory elements enable a skilled artisan to use sequences encoding a protein of the invention as an investigative tool in sense (Youssoufian H and H F Lodish 1993 Mol Cell Biol 13:98-104) or antisense (Eguchi et al (1991) Annu Rev Biochem 60:631-652) regulation of gene function. Such technology is well known in the art, and sense or antisense oligomers, or larger fragments, can be designed from various locations along the coding or control regions.

Genes encoding a protein of the invention can be turned off by transfecting a cell or tissue with vectors which express high levels of a desired BPR-encoding fragment. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA, to the regulatory regions of a gene encoding a protein of the invention, i.e. the promoters, enhancers, and introns. Preferably, oligonucleotides are derived from the transcription initiation site, eg, between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a protein of the invention.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

An antibody against a BPR Related Protein may be conjugated to chemotherapeutic drugs, toxins, immunological response modifiers, hematogenous agents, enzymes, and radioisotopes and used in the prevention and treatment of cancer. For example, an antibody against a BPR Related Protein may be conjugated to toxic moieties including but not limited to ricin A, diphtheria toxin, abrin, modeccin, or bacterial toxins from Pseudomonas or Shigella. Toxins and their derivatives have been reported to form conjugates with antibodies specific to particular target tissues, such as cancer or tumor cells in order to obtain specifically targeted cellular toxicity (Moolten F. L. et al, Immun. Rev. 62:47-72, 1982, and Bernhard, M. I. Cancer Res. 43:4420, 1983).

Conjugates can be prepared by standard means known in the art. A number of bifunctional linking agents (e.g. heterobifunctional linkers such as N-succinimidyl-3-(2-pyridyldithio)propionate) are available commercially from Pierce Chemically Company, Rockford, Ill.

Administration of the antibodies or immunotoxins for therapeutic use may be by an intravenous route, although with proper formulation additional routes of administration such as intraperitoneal, oral, or transdermal administration may also be used.

A BPR Related Protein may be conjugated to chemotherapeutic drugs, toxins, immunological response modifiers, enzymes, and radioisotopes using methods known in the art.

The invention also provides immunotherapeutic approaches for preventing or reducing the severity of a cancer. The clinical signs or symptoms of the cancer in a subject are indicative of a beneficial effect to the patient due to the stimulation of the subject's immune response against the cancer. Stimulating an immune response refers to inducing an immune response or enhancing the activity of immunoeffector cells in response to administration of a vaccine preparation of the invention. The prevention of a cancer can be indicated by an increased time before the appearance of cancer in a patient that is predisposed to developing cancer due for example to a genetic disposition or exposure to a carcinogenic agent. The reduction in the severity of a cancer can be indicated by a decrease in size or growth rate of a tumor.

Vaccines can be derived from a BPR Related Protein, peptides derived therefrom, or chemically produced synthetic peptides, or any combination of these molecules, or fusion proteins or peptides thereof. The proteins, peptides, etc. can be synthesized or prepared recombinantly or otherwise biologically, to comprise one or more amino acid sequences corresponding to one or more epitopes of a tumor associated protein. Epitopes of a tumor associated protein will be understood to include the possibility that in some instances amino acid sequence variations of a naturally occurring protein or polypeptide may be antigenic and confer protective immunity against cancer or anti-tumorigenic effects. Sequence variations may include without limitation, amino acid substitutions, extensions, deletions, truncations, interpolations, and combinations thereof. Such variations fall within the scope of the invention provided the protein containing them is immunogenic and antibodies against such polypeptide cross-react with naturally occurring BPR Related Protein to a sufficient extent to provide protective immunity and/or anti-tumorigenic activity when administered as a vaccine.

The proteins, peptides etc, can be incorporated into vaccines capable of inducing an immune response using methods known in the art. Techniques for enhancing the antigenicity of the proteins, peptides, etc. are known in the art and include incorporation into a multimeric structure, binding to a highly immunogenic protein carrier, for example, keyhole limpet hemocyanin (KLH), or diptheria toxoid, and administration in combination with adjuvants or any other enhancer of immune response.

Vaccines may be combined with physiologically acceptable media, including immunologically acceptable diluents and carriers as well as commonly employed adjuvants such as Freund's Complete Adjuvant, saponin, alum, and the like.

It will be further appreciated that anti-idiotype antibodies to antibodies to BPR Related Proteins described herein are also useful as vaccines and can be similarly formulated.

The administration of a vaccine in accordance with the invention, is generally applicable to the prevention or treatment of cancers including breast and testicular cancer.

The administration to a patient of a vaccine in accordance with the invention for the prevention and/or treatment of cancer can take place before or after a surgical procedure to remove the cancer, before or after a chemotherapeutic procedure for the treatment of cancer, and before or after radiation therapy for the treatment of cancer and any combination thereof. The cancer immunotherapy in accordance with the invention would be a preferred treatment for the prevention and /or treatment of cancer, since the side effects involved are substantially minimal compared with the other available treatments e.g. surgery, chemotherapy, radiation therapy. The vaccines have the potential or capability to prevent cancer in subjects without cancer but who are at risk of developing cancer.

The activity of the proteins, substances, compounds, antibodies, nucleic acid molecules, agents, and compositions of the invention may be confirmed in animal experimental model systems. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

4.4 Other Applications

The nucleic acid molecules disclosed herein may also be used in molecular biology techniques that have not yet been developed, provided the new techniques rely on properties of nucleotide sequences that are currently known, including but not limited to such properties as the triplet genetic code and specific base pair interactions.

The invention also provides methods for studying the function of a polypeptide of the invention. Cells, tissues, and non-human animals lacking in expression or partially lacking in expression of a nucleic acid molecule or gene of the invention may be developed using recombinant expression vectors of the invention having specific deletion or insertion mutations in the gene. A recombinant expression vector may be used to inactivate or alter the endogenous gene by homologous recombination, and thereby create a deficient cell, tissue, or animal.

Null alleles may be generated in cells, such as embryonic stem cells by deletion mutation. A recombinant gene may also be engineered to contain an insertion mutation that inactivates the gene. Such a construct may then be introduced into a cell, such as an embryonic stem cell, by a technique such as transfection, electroporation, injection etc. Cells lacking an intact gene may then be identified, for example by Southern blotting, Northern Blotting, or by assaying for expression of the encoded protein using the methods described herein. Such cells may then be fused to embryonic stem cells to generate transgenic non-human animals deficient in a protein of the invention. Germline transmission of the mutation may be achieved, for example, by aggregating the embryonic stem cells with early stage embryos, such as 8 cell embryos, in vitro; transferring the resulting blastocysts into recipient females and; generating germline transmission of the resulting aggregation chimeras. Such a mutant animal may be used to define specific cell populations, developmental patterns and in vivo processes, normally dependent on gene expression.

The invention thus provides a transgenic non-human mammal all of whose germ cells and somatic cells contain a recombinant expression vector that inactivates or alters a gene encoding a BPR Related Protein. In an embodiment the invention provides a transgenic non-human mammal all of whose germ cells and somatic cells contain a recombinant expression vector that inactivates or alters a gene encoding a BPR Related Protein resulting in a BPR Related Protein associated pathology. Further the invention provides a transgenic non-human mammal which does not express or partially expresses a BPR Related Protein of the invention. In an embodiment, the invention provides a transgenic non-human mammal which doe not express or partially expresses, a BPR Related Protein of the invention resulting in a BPR Related Protein associated pathology. A BPR Related Protein pathology refers to a phenotype observed for a BPR Related Protein homozygous or heterozygous mutant.

A transgenic non-human animal includes but is not limited to mouse, rat, rabbit, sheep, hamster, dog, cat, goat, and monkey, preferably mouse.

The invention also provides a transgenic non-human animal assay system which provides a model system for testing for an agent that reduces or inhibits a pathology associated with a BPR Related Protein, preferably a BPR Related Protein associated pathology, comprising:

(a) administering the agent to a transgenic non-human animal of the invention; and

(b) determining whether said agent reduces or inhibits the pathology (e.g. BPR Related Protein associated pathology) in the transgenic non-human animal relative to a transgenic non-human animal of step (a) which has not been administered the agent.

The agent may be useful in the treatment and prophylaxis of conditions such as cancer as discussed herein. The agents may also be incorporated in a pharmaceutical composition as described herein.

The following non-limiting examples are illustrative of the present invention:

### EXAMPLE

Materials and Methods

Cloning of the BPR Gene

In the search for genes involved in the process of apoptosis, an analysis was performed for different conserved motifs of the apoptosis-related genes (BH1, BH2, BH3, and BH4) using the TBLASTN program, against the unfinished High Throughput Genomic Sequences database (htgs). A potential BH2 domain was identified in the BAC clone BC42053, sequenced by the Lawrence Livermore National Laboratory (LLNL). Genomic sequences from this clone, were in the form of 87 contigs of different lengths. The clone containing this gene was obtained from the Lawrence Livermore National Laboratory (LLNL), and the genomic DNA was isolated. The chromosome 19 EcoRl restriction map (21) and long PCR strategies, using genomic DNA from this clone, were used to construct a contiguous area of the genomic area of interest. Bioinformatic approaches were used, as previously described (22-24), to predict the presence of new genes and a putative new appoptosis-related, proline-rich protein was identified. Blast search and EcoRl restriction digestion analysis of the sequence of one adjacent BAC clone (R31181) allowed for the identification of the relative position of the new gene and other previously identified genes; RRAS, IRF3 and PRMT1, along the same chromosomal region (19q13.3). The sequence of the putative new gene was then verified by different experimental approaches including sequencing, EST database search and RT-PCR screening of tissues, as described below.

Cloning and Sequencing of Genomic PCR Products

Positive genomic fragments as well as long PCR products were gel-purified, cloned into the TOPO-XL cloning vector and propagated in LB medium with ampicillin. Plasmids were then purified and sequenced as previously described (25).

Expressed Sequence Tag (EST) Search

The predicted exons of the putative new gene were subjected to homology search using the BLASTN algorithm (26) against the human EST database (dbEST). Clones with >98% homology (Table 1) were obtained from the I.M.A.G.E. consortium (27) through Research Genetics Inc, Huntsville, Ala. The clones were propagated, purified (28) and sequenced from both directions with an automated sequencer, using insert-flanking vector primers.

Reverse Transcriptase Polymerase Chain Reaction (RT-PCR) for the BPR Gene

2 μg of total RNA from different human tissues (Clontech, Palo Alto, Calif.) were reverse-transcribed into first strand cDNA using the Superscript™ preamplification system (Gibco BRL Gaithersburg, Md.). The final volume was 20 μl. Based on the combined information obtained from the predicted genomic structure of the new gene and the EST sequences, two gene-specific primers were designed (BPR-F1 and BPR-R7, SEQ ID Nos. 9 and 16, respectively) (Table 2) and PCR was carried out in a reaction mixture containing 1 μl of cDNA, 10 mM Tris-HCl (pH 8.3), 50 mM KCl, 1.5 mM MgCl₂, 200 μM dNTPs (deoxynucleoside triphosphates), 150 ng of primers and 2.5 units of HotStar™ DNA polymerase (Qiagen Inc., Valencia, Calif.) on a Perkin-Elmer 9600 thermal cycler. The cycling conditions were 95° C. for 15 minutes to activate the Taq DNA polymerase, followed by 35 cycles of 94° C. for 30 s, 62° C. for 30 s, 72° C. for 1 min and a final extension step at 72° C. for 10 min. Equal amounts of PCR products were electrophoresed on 2% agarose gels and visualized by ethidium bromide staining.

5′ and 3′ Rapid Amplification of cDNA Ends (5′ and 3′ RACE)

According to the EST sequences and the predicted structure of the BPR gene, four reverse gene specific primers were designed (BPR-R2, BPR-R3 and BPR-R6, BPR-R7, SEQ ID Nos. 13, 14, 12, and 10, respectively). Two rounds of RACE reactions (nested PCR) were performed with 5 μl Marathon Ready™ cDNA from human thymus (Clontech) as a template. The reaction mix and PCR conditions were selected according to the manufacturer's recommendations. In brief, the initial denaturation was for 5 min at 94° C., followed by 94° C. for 5 s and 72° C. for 2 min, for 5 cycles; then, 94° C. for 5 s and 68° C. for 2 min, for 5 cycles; then, 94° C. for 5 s and 65° C. for 2 min for 30 cycles for the first reaction and 25 cycles for the nested PCR reaction. Positive bands were gel-purified using Qiagen Gel Purification kit according to manufacturer's recommendations.

Cloning and Sequencing of the PCR Products

To verify the identity of the PCR products, they were cloned into the pCR 2.1-TOPO vector (Invitrogen, Carlsbad, Calif., USA) according to the manufacturer's instructions. The inserts were sequenced from both directions using vector-specific primers, with an automated DNA sequencer.

Tissue Expression

Total RNA isolated from 28 different human tissues was purchased from Clontech. cDNA was prepared as described above and used for PCR reactions. Tissue cDNAs were amplified at various dilutions using two gene-specific primers (BPR-F2 and BPR-R6, SEQ ID NOs. 11 and 12, respectively) (Table 2). The same cDNAs were used for actin gene amplification (control). PCR products were cloned and sequenced.

Structure Analysis of the BPR Protein

Multiple alignment was performed using the “Clustal X” software package (29) and the multiple alignment program available from the Baylor College of Medicine, Houston, Tex., USA. Hydrophobicity study was performed using the Baylor College of Medicine search launcher. Signal peptide was predicted using the “SignalP” server (30). Protein structure analysis was performed by “SAPS” (structural analysis of protein sequence) program (31). Sequence analysis tools were utilized to detect the presence of possible sites of post-translational modification on the BPR protein. The analysis program PROSITE (32) and NetOGlyc 2.0 (33) were used to detect N- and O-glycosylation, as well as the presence of kinase phosphorylation motifs.

Results

Cloning of the BPR Gene

In order to investigate the presence of new apoptosis related genes, a BAC clone (BC42053) was identified and processed as described above. The putative new gene sequence, predicted with bioinformatics, was then blasted against the human EST database and seven EST clones were found with >98% identity with one or more predicted exons (Table 1). Two clones were 99% identical to the last exon and the 3′ untranslated region of the gene. These ESTs contained stretches of 17 or 15 adenine (A) nucleotides, respectively, that were not found in the genomic sequence. 3′ RACE reactions and sequencing were also performed to verify the 3′ end of the gene and the position of the poly A tail.

To identify the full mRNA structure of the gene and to determine the exon/intron boundaries, PCR reactions were performed using primers located in different predicted exons, using a panel of 28 human tissue cDNAs as templates. PCR products were purified and sequenced. Two of these primers (BPR-F1 and BPR-R7, SEQ ID NOs. 9 and 10, respectively) (Table 2) were able to amplify the full coding region of the gene from different tissues. Comparing the MRNA with the genomic structure indicated the presence of a gene formed of seven coding exons with 6 intervening introns. 5′ RACE reactions and sequencing were performed in order to obtain the 5′ end of the gene and a 660 bp of sequence was identified upstream to BPR-F1 primer. A putative TATA box is presented 29 bp upstream from the first nucleotide of the 5′ RACE product. An in-frame methionine start codon was found in the first exon that matches well with the consensus Kozak sequence (GCCA/GCCATGG, SEQ. ID. NO. 28) (34). Translation of the mRNA sequence in all possible reading frames revealed the presence of only one frame that gives an uninterrupted polypeptide chain, which also contains a highly conserved BH2 domain present of the Bcl-2 family of genes and eight PXXP motifs, as discussed below.

Structural Characterization of the BPR Gene

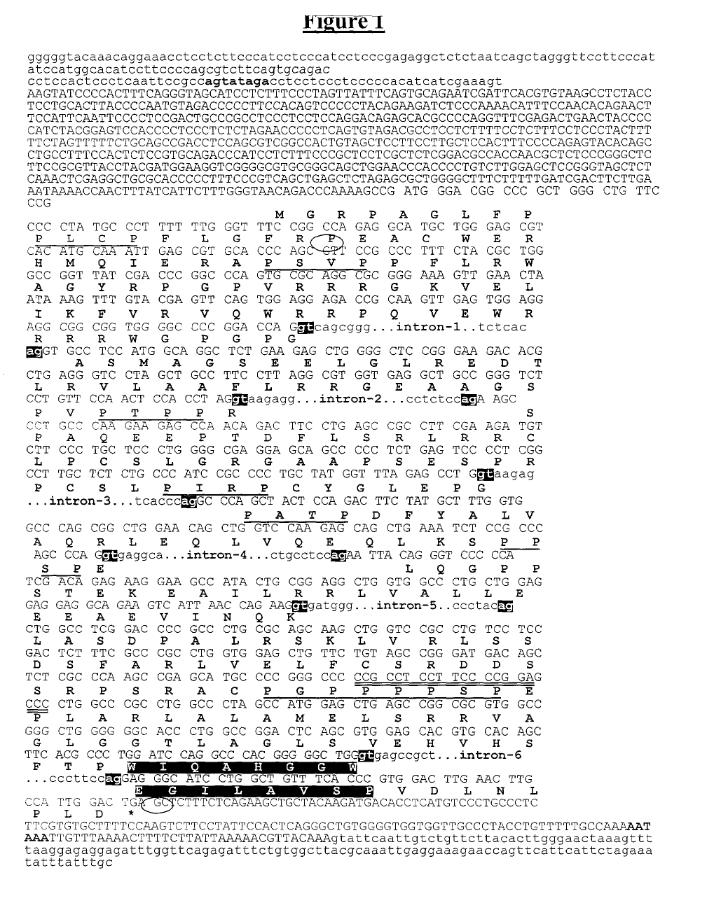

As shown in FIG. 1, the BPR gene is formed of 7 coding exons and 6 intervening introns, spanning an area of 8,772 bp of genomic sequence on chromosome 19q13.3. All of the exon/intron splice sites (mGT . . . AGm) conform to the consensus sequence for eukaryotic splice sites (35). Lengths of the coding exons are 922, 115, 143, 87, 92, 273 and 216 bp, respectively. The predicted protein-coding region of the gene is formed of 1005 bp, encoding a deduced 334 amino acid polypeptide with a predicted molecular weight of 36.8 kDa and isoelectric point of 9.45.

Nucleotides 8987-8992 (AATAAA, SEQ ID NO. 29) (GenBank submission Accession #AF289220) are identical with a consensus polyadenylation signal (36) and are followed, after 35 nucleotides, by the poly A tail not found in the genomic sequence. Another potential polyadenylation signal (ATTAAA, SEQ ID NO.30) was discernible in the 3′ untranslated region, 11 bases upstream of the poly A tail. Although AATAAA is highly conserved, natural variants do occur, and the ATTAAA sequence is reported to occur as a natural polyadenylation variant in 12% of vertebrate mRNA sequences (37).

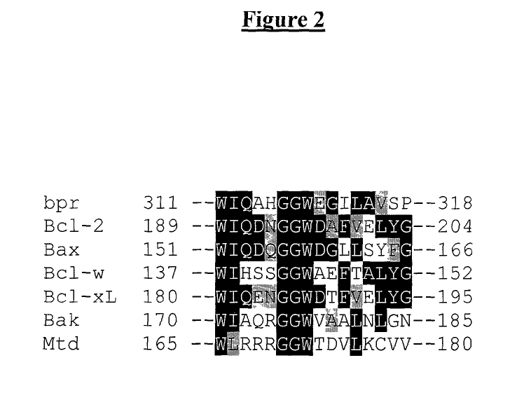

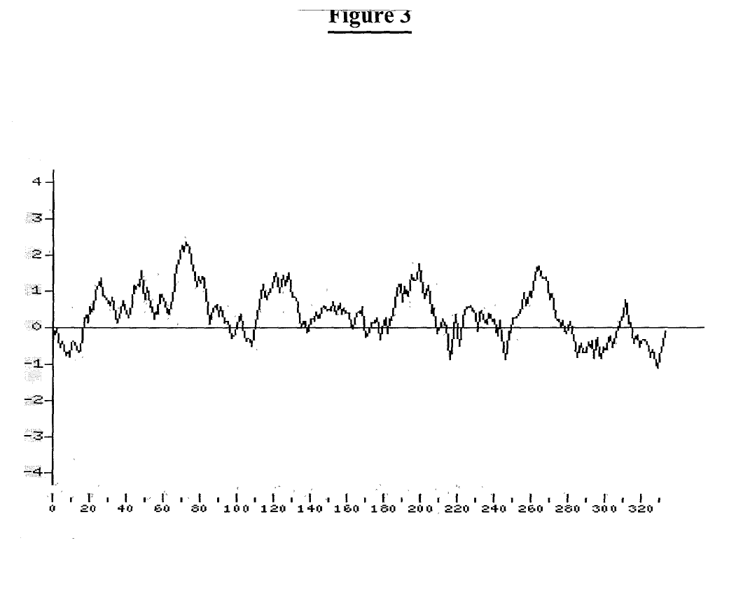

The highlighted region in FIG. 1 indicates an 16-amino acid sequence, found in the BH2 domain of several members of the Bcl-2 family of proteins (7). The amino acid loop (WIQXXGGW—SEQ ID NO 26) at positions 311-318 was also found in Bcl-2, Bax and Bcl-X_{L} proteins (FIG. 2). No BH1, BH3 and BH4 domains were found. Bcl-2 family genes containing only one BH domain have been reported (38). Comparative analysis revealed that the BPR protein sequence has a low degree of homology with other members of Bcl-2 multigene family. BPR shows 20% protein identity with the Bcl-2 and Bcl-w proteins, 18% with the Mtd protein and 18% identity with the Bcl-X_{L}, Bax and Bak proteins. This is not surprising since the apoptosis related proteins have low homology (7). Hydrophobicity analysis revealed a slightly hydrophobic N-terminal region (FIG. 3), consistent with the possibility that this region has no signal sequence. Software analysis of the BPR protein sequence did not predict any signal peptide cleavage site. Through the use of sequence analysis tools, various putative post-translational modification sites were identified (Table 3). There are numerous potential sites for O-glycosylation. Furthermore, several possible sites of phosphorylation have been identified for cAMP-dependent protein kinase, protein kinase C, and casein kinase 2. In addition, several N-myristoylation sites have been predicted (Table 3).

BPR protein was found to have proline rich sites. One PPPP site as well as five PP amino acid sites are present in this protein. Eight putative PXXP motifs were also identified (FIG. 1). It is known that SH3 domains recognize proline-rich sequences and all known SH3-binding proteins contain proline-rich regions with at least one PXXP motif (39, 40). Moreover, the amino acid loop (PPSPEP) at positions 271-276 of the BPR protein is identical with the PXXP motif present in the RRAS and TC21 oncogenes (19). This motif is required for integrin activation.

Splice Variants of the BPR Gene

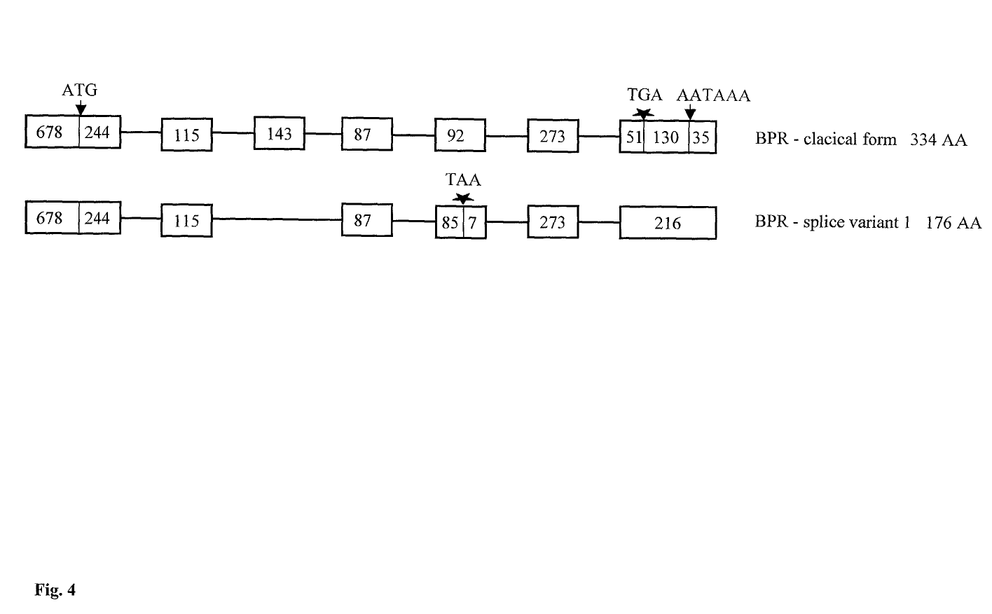

PCR screening for BPR transcripts using gene-specific primers (BPR-F1 and BPR-R7) (Table 1) revealed the presence of 2 bands in most of the tissue cDNAs examined. The two bands were gel purified, cloned and sequenced. The upper band represents the classical form of the gene, and the lower band is splice variant 1. As shown in FIG. 4 this variant (BPR-splice variant 1) is missing the exon 3 (143 bp). This splice variant is expected to encode for a truncated protein of 176 amino acids with five PP proline sites, two putative PXXP motifs and without BH2 homology domain.

Mapping of the BPR Gene

Restriction analysis of overlapping BAC clones spanning the chromosomal area of interest, as well as alignment strategies (26) allowed us to locate a number of previously identified genes, together with the newly identified gene, along the EcoRl restriction map of the area (21). By identifying the position of the RRAS, IRF3, PRMT1/HRMT1L2 and BPR genes along these clones, the relative location and the direction of transcription of these four genes was precisely defined.

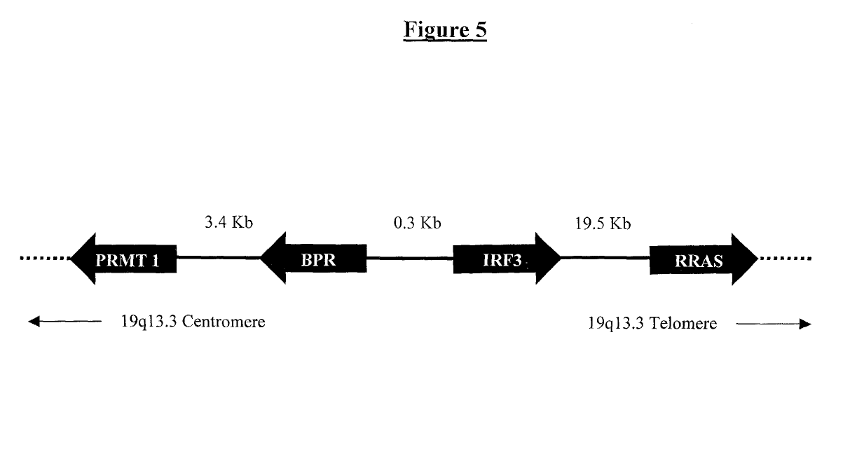

PRMT1 is the most centromeric and its direction of transcription is from telomere to centromere, followed by BPR, which is more telomeric and transcribes in the same direction. The distance between the two genes is 3.4 Kb. RRAS and IRF3 genes are more telomeric, located at a distance of 23.9 and 0.3 Kb from BPR, respectively, and are transcribed in the opposite direction (FIG. 5).

Expression Patterns of BPR

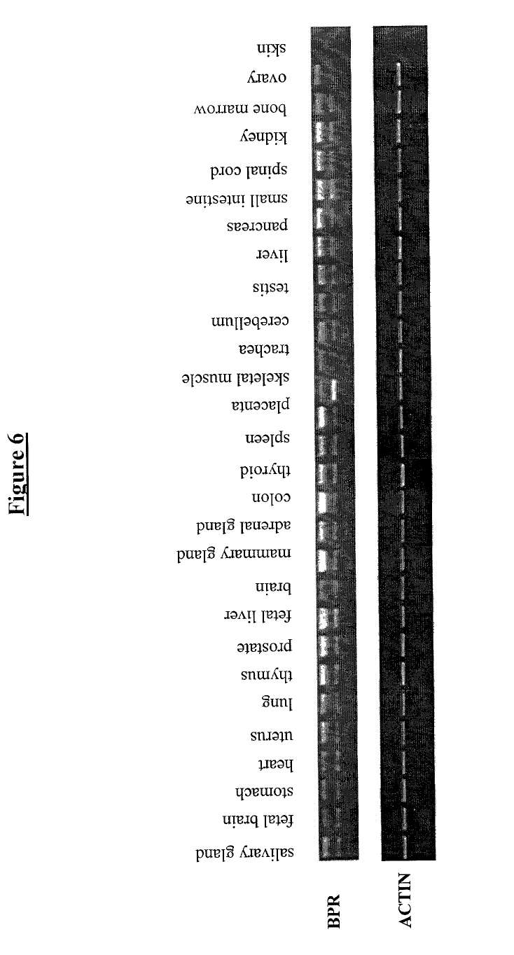

RT-PCR analysis, with primers BPR-F2 (SEQ ID NO. 11) and BPR-R6 (SEQ ID NO. 12), from different human tissues were used to identify the expression pattern of the BPR. Actin was used as a control gene. In each of the 26 adult and 2 fetal tissues tested, a BPR transcript was identified. As shown in FIG. 6, the classic form of the BPR gene is highly expressed in the thymus, prostate, fetal liver, mammary, colon, placenta, small intestine, kidney and bone marrow. Lower levels of expression are also seen in all other tissues tested. The splice variant 1 is highly expressed in skeletal muscle, fetal liver and spinal cord. In skeletal muscle, the levels of variant 1 transcripts are higher than the classic form, in contrast to other tissues. In order to verify the RT-PCR specificity, representative PCR products were cloned and sequenced. These results were reproducible with different sets of primers and different reaction conditions.

Differential Expression of the BPR Gene in Testicular and Breast Cancer Tissues

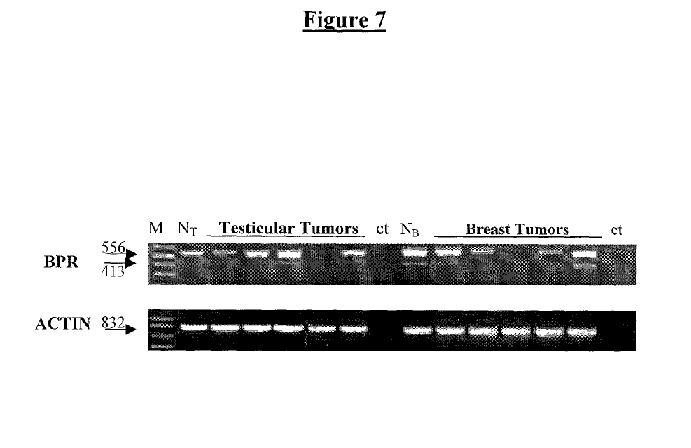

To characterize the extent and frequency of expression of the BPR gene in testicular and breast tumors, RT-PCR was used with cDNA derived from normal and malignant tissues. Of the 20 testicular cancer cDNAs tested, the classic form of BPR was low or undetectable in seven tumor tissues, increased in six tumor tissues, and remained unchanged, relative to the normal testicular tissue, in seven tumors. BPR-variant 1 expression was also quite variable between the tested tumors with no consistent pattern. (FIG. 7).

In the breast, the classic form is not expressed at all in 4 out of 25 tested tumors. High levels of the BPR transcript were observed in the twelve tumors (FIG. 7). BPR-variant 1 was not expressed in 3 tumors and equivalent expressed in the rest of the tested tumors. These results were reproducible using two set of primers (BPR-F2 and BPR-R6, SEQ ID NOs., 11, and 12 respectively, BPR-F 1 and BPR-R6, SEQ ID NOs., 9, and 12 respectively) and different reaction conditions. Actin was the control gene. As mentioned above, BPR was also found to have a BH2 homology domain as well as five PXXP motifs. Taken together, these preliminary results suggest that this gene may be related to testicular and breast cancer pathogenesis.

Discussion

A novel human gene, BPR, has been cloned which maps to chromosome 19q.13.3-q13.4. Bioinformatic approaches and EST analysis were first used to delineate the genomic organization of the gene and predict the putative mRNA coding region. RT-PCR and sequencing were used verify the exons and the splice junctions. The 3′ and 5′ end of the gene were verified using RACE technology. Present at the 3′-terminus of two ESTs was also a poly-A tail. The BPR gene is formed of 7 coding exons and 6 intervening introns. One alternative splice variant, missing exon 3 was also identified.

The new gene is localized in an area that contains a number of well studied genes such as the RRAS, IRF3 and PRMT1/HRMT1L2. The RRAS gene encodes a small GTPase (41,42). Although its function is not fully understood, it has been shown to promote integrin activity and cell adhesion (19, 43). The IRF3 gene encodes a protein which binds to the interferon stimulated response element and activates expression of interferon-stimulated genes (44,45). The PRMT1 gene encodes an arginine methyltransferase which has been shown to reduce the antiproliferative effect of interferon (46, 47). The apoptosis regulator gene BAX is also located in chromosome 19q13.3 (48, 49). These genes have been shown to be involved in malignancy, directly (e.g. RRAS) or indirectly.

The BPR gene is constitutively expressed in all tested tissues suggesting that the encoded protein serves an important function in many, if not all cell types.

The predicted 36.8 kDa BPR protein contained several small proline-rich motifs (PXXP). Similar motifs have been shown to interact with the SH3 domain of the c-Src and c-Abl protooncoproteins (50, 51). A PPSP motif present in the C-terminal portion of the protein has also been identified as a functional element in the TC21 protein, a member of the RRAS superfamily (19). Recently, another protooncoprotein, RRAS, was also shown to contain a proline-rich domain. The presence of the proline-rich area of the RRAS protein has been shown to mediate binding to the SH3 domains of integrins, a protein family which is involved in cell adhesion to extracellular matrix domains (19). Thus, the proline-rich regions appear to be candidates for activation of at least some integrin molecules. Although the PXXP motif is important in mediating the interaction with the SH3 domains, other sequences have been shown to confer interaction specificity (20).

The BPR protein showed a ˜20% homology to two members of the anti-apoptotic protein family, the Bcl-2 and Mtd proteins as well as the Bax, Bcl-W, Bak and Bcl-X_{L} proteins. Although the degree of homology was very low, this is not unusual since other members of the Bcl-2 family such as the Bcl-2 and Bax, also share only 25% homology. In addition, the predicted protein contains the WIQXXGGW (SEQ ID NO. 26) motif which constitutes the BH2 domain, but lacks the BH1, BH3 or BH4 domains that are present in other members of the family. The BH2 domain is present in the anti-apoptotic proteins Bcl-2 and Bcl-X_{L} and is important in the homo- or heterodimerization of the family members (3, 52-54). Although most Bcl-2 family members contain a combination of the BH1, BH2, BH3 or BH4 domains, one member of the family, a truncated form of the Bax protein, contains only one BH3 domain (55). Mammalian activator of apoptosis, Harakiri, also contains a BH3 domain, but no BH1, BH2 or BH4 domains (38). In addition, the carbohydrate-binding protein, Galectin3, was recently shown to contain only one BH1 domain. Yet, the protein does display an anti-apoptotic activity (56). Thus, it appears that a full complement of BH domains may not be necessary to confer apoptotic or anti-apoptotic activity. The BH2 domain of the BPR protein may also participate in interactions with other apoptotic proteins. In malignancies, such as testicular and breast tumors, interactions between the various members of the Bcl-2 family have been shown to modulate apoptotic events that can regulate response to chemotherapy (9, 10, 12). Examination of the BPR mRNA levels in testicular and breast cancer, showed that the BPR levels in carcinomas vary, relative to the levels observed in normal tissue. Changes in the observed BPR mRNA levels in testicular and breast cancer suggest that BPR may be involved in the events determining the apoprotic behavior of the cells.

Both the BH2 and the proline-rich domains play important roles in the productive interaction of proteins during events such as apoptosis or its repression, epidermal factor signaling, cellular localization of cytoplasmic proteins and activation of the phosphatidylinositol 3-kinase in response to IgM crosslinking (17, 18, 57, 58). The presence of the BH2 domain, present in most anti-apoptotic proteins, together with the proline-rich motifs, suggests that the BPR protein may participate in protein-protein interactions. This is the first gene identified, encoding for a protein which contains both a proline-rich and a BH2 domain. The presence of both domains may represent the cross-road between proteins involved in the regulation of apoptotic events and proteins containing SH3 domains.

Having illustrated and described the principles of the invention in a preferred embodiment, it should be appreciated to those skilled in the art that the invention can be modified in arrangement and detail without departure from such principles. All modifications coming within the scope of the following claims are claimed.

All publications, patents and patent applications referred to herein are incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Wyllie, A. H. Apoptosis (the 1992 Frank Rose Memorial Lecture). Br J Cancer. 67. 205-8, 1993.

2. Choi, W. S., Yoon, S. Y., Chang, II, Choi, E. J., Rhim, H., Jin, B. K., Oh, T. H., Krajewski, S., Reed, J. C., and Oh, Y. J. Correlation between structure of Bcl-2 and its inhibitory function of JNK and caspase activity in dopaminergic neuronal apoptosis. J Neurochem. 74: 1621-6, 2000.

3. Zha, H., Aime-Sempe, C., Sato, T., and Reed, J. C. Proapoptotic protein Bax heterodimerizes with Bcl-2 and homodimerizes with Bax via a novel domain (BH3) distinct from BH1 and BH2. J Biol Chem. 271. 7440-4, 1996.

4. Martin, S. J. and Green, D. R. Protease activation during apoptosis: death by a thousand cuts? Cell. 82: 349-52, 1995.

5. Thornberry, N. A. and Lazebnik, Y. Caspases: enemies within. Science. 281: 1312-6,1998.

6. Adams, J. M. and Cory, S. The Bcl-2 protein family: arbiters of cell survival. Science. 281: 1322-6, 1998.

7. Reed, J. C., Zha, H., Aime-Sempe, C., Takayama, S., and Wang, H. G. Structure-function analysis of Bcl-2 family proteins. Regulators of programmed cell death. Adv Exp Med Biol. 406: 99-112, 1996.

8. Korsmeyer, S. J. BCL-2 gene family and the regulation of programmed cell death. Cancer Res. 59: 1693s-1700s, 1999.

9. Chresta, C. M., Arriola, E. L., and Hickman, J. A. Apoptosis and cancer chemotherapy. Behring Inst Mitt 232-40, 1996.

10. Arriola, E. L., Rodriguez-Lopez, A. M., Hickman, J. A., and Chresta, C. M. Bcl-2 overexpression results in reciprocal downregulation of Bcl-X(L) and sensitizes human testicular germ cell tumours to chemotherapy-induced apoptosis. Oncogene. 18:1457-64, 1999.

11. Amin, F., Bowen, I. D., Szegedi, Z., Mihalik, R., and Szende, B. Apoptotic and non-apoptotic modes of programmed cell death in MCF-7 human breast carcinoma cells. Cell Biol Int. 24: 253-60, 2000.

12. Zhang, G. J., Kimijima, I., Onda, M., Kanno, M., Sato, H., Watanabe, T., Tsuchiya, A., Abe, R., and Takenoshita, S. Tamoxifen-induced apoptosis in breast cancer cells relates to down-regulation of bcl-2, but not bax and bcl-X(L), without alteration of p53 protein levels. Clin Cancer Res. 5: 2971-7, 1999.

13. Takayama, S., Sato, T., Krajewski, S., Kochel, K., Irie, S., Millan, J. A., and Reed, J. C. Cloning and functional analysis of BAG-1: a novel Bcl-2-binding protein with anti-cell death activity. Cell. 80: 279-84, 1995.

14. Wang, H. G., Miyashita, T., Takayama, S., Sato, T., Torigoe, T., Krajewski, S., Tanaka, S., Hovey, L., 3rd, Troppmair, J., Rapp, U. R., and et al. Apoptosis regulation by interaction of Bcl-2 protein and Raf-1 kinase. Oncogene. 9: 2751-6, 1994.

15. Iwahashi, H., Eguchi, Y., Yasuhara, N., Hanafusa, T., Matsuzawa, Y., and Tsujimoto, Y. Synergistic anti-apoptotic activity between Bcl-2 and SMN implicated in spinal muscular atrophy. Nature. 390: 413-7, 1997.

16. Lefebvre, S., Burglen, L., Reboullet, S., Clermont, O., Burlet, P., Viollet, L., Benichou, B., Cruaud, C., Millasseau, P., Zeviani, M., and et al. Identification and characterization of a spinal muscular atrophy-determining gene. Cell. 80: 155-65, 1995.

17. Lowenstein, E. J., Daly, R. J., Batzer, A. G., Li, W., Margolis, B., Lammers, R., Ullrich, A., Skolnik, E. Y., Bar-Sagi, D., and Schlessinger, J. The SH2 and SH3 domain-containing protein GRB2 links receptor tyrosine kinases to ras signaling. Cell. 70: 431-42, 1992.

18. Bar-Sagi, D., Rotin, D., Batzer, A., Mandiyan, V., and Schlessinger, J. SH3 domains direct cellular localization of signaling molecules. Cell. 74: 83-91, 1993.

19. Wang, B., Zou, J. X., Ek-Rylander, B., and Ruoslahti, E. R-Ras contains a proline-rich site that binds to SH3 domains and is required for integrin activation by R-Ras. J Biol Chem. 275. 5222-7, 2000.

20. Alexandropoulos, K., Cheng, G., and Baltimore, D. Proline-rich sequences that bind to Src homology 3 domains with individual specificities. Proc Natl Acad Sci U S A. 92: 3110-4,1995.

21. Ashworth, L. K., Batzer, M. A., Brandriff, B., Branscomb, E., de Jong, P., Garcia, E., Garnes, J. A., Gordon, L. A., Lamerdin, J. E., Lennon, G., and et al. An integrated metric physical map of human chromosome 19. Nat Genet. 11: 422-7, 1995.

22. Bonet, M., Steel, M., Warnow, T., and Yooseph, S. Better methods for solving parsimony and compatibility. J Comput Biol. 5: 391-407, 1998.

23. Smith, D. K. and Xue, H. A major component approach to presenting consensus sequences. Bioinformatics. 14: 151-6, 1998.

24. Murakami, K. and Takagi, T. Gene recognition by combination of several gene-finding programs. Bioinformatics. 14: 665-75, 1998.

25. Sanger, F., Nicklen, S., and Coulson, A. R. DNA sequencing with chain-terminating inhibitors. Proc Natl Acad Sci U S A. 74: 5463-7, 1977.

26. Altschul, S. F., Madden, T. L., Schaffer, A. A., Zhang, J., Zhang, Z., Miller, W., and Lipman, D. J. Gapped BLAST and PSI-BLAST: a new generation of protein database search programs. Nucleic Acids Res. 25: 3389-402, 1997.

27. Lennon, G., Auffray, C., Polymeropoulos, M., and Soares, M. B. The I.M.A.G.E. Consortium: an integrated molecular analysis of genomes and their expression. Genomics. 33: 151-2, 1996.

28. Sambrook, J., Fritsch, E. F., and Maniatis, T. Molecular Cloning: A Laboratory Manual. NY: Cold Sping Harbor Laboratory, 1989.

29. Aiyar, A. The use of CLUSTAL W and CLUSTAL X for multiple sequence alignment. Methods Mol Biol. 132: 221-41, 2000.

30. Nielsen, H., Brunak, S., and von Heijne, G. Machine learning approaches for the prediction of signal peptides and other protein sorting signals. Protein Eng. 12: 3-9, 1999.

31. Brendel, V., Bucher, P., Nourbakhsh, I. R., Blaisdell, B. E., and Karlin, S. Methods and algorithms for statistical analysis of protein sequences. Proc Natl Acad Sci U S A. 89: 2002-6, 1992.

32. Bairoch, A., Bucher, P., and Hofmann, K. The PROSITE database, its status in 1997. Nucleic Acids Res. 25. 217-21, 1997.

33. Hansen, J. E., Lund, O., Tolstrup, N., Gooley, A. A., Williams, K. L., and Brunak, S. NetOglyc: prediction of mucin type O-glycosylation sites based on sequence context and surface accessibility. Glycoconj J. 15: 115-30, 1998.

34. Kozak, M. An analysis of vertebrate mRNA sequences: intimations of translational control. J Cell Biol. 115: 887-903, 1991.

35. Iida, Y. Quantification analysis of 5′-splice signal sequences in mRNA precursors. Mutations in 5′-splice signal sequence of human beta-globin gene and beta-thalassemia. J Theor Biol. 145: 523-33, 1990.

36. Proudfoot, N. J., Cheng, C. C., and Brownlee, G. G. Sequence analysis of eukaryotic mRNA. Prog Nucleic Acid Res Mol Biol. 19: 123-34, 1976.

37. Sheets, M. D., Ogg, S. C., and Wickens, M. P. Point mutations in AAUAAA and the poly (A) addition site: effects on the accuracy and efficiency of cleavage and polyadenylation in vitro. Nucleic Acids Res. 18: 5799-805, 1990.

38. Inohara, N., Ding, L., Chen, S., and Nunez, G. harakiri, a novel regulator of cell death, encodes a protein that activates apoptosis and interacts selectively with survival-promoting proteins Bcl-2 and Bcl-X(L). Embo J. 16: 1686-94, 1997.

39. Sparks, A. B., Rider, J. E., Hoffinan, N. G., Fowlkes, D. M., Quillam, L. A., and Kay, B. K. Distinct ligand preferences of Src homology 3 domains from Src, Yes, Abl, Cortactin, p53bp2, PLCgamma, Crk, and Grb2. Proc Natl Acad Sci U S A. 93: 1540-4, 1996.

40. Cohen, G. B., Ren, R., and Baltimore, D. Modular binding domains in signal transduction proteins. Cell. 80: 237-48, 1995.

41. Zhang, Z., Vuori, K., Wang, H., Reed, J. C., and Ruoslahti, E. Integrin activation by R-ras. Cell. 85: 61-9, 1996.

42. Wang, H. G., Millan, J. A., Cox, A. D., Der, C. J., Rapp, U. R., Beck, T., Zha, H., and Reed, J. C. R-Ras promotes apoptosis caused by growth factor deprivation via a Bcl-2 suppressible mechanism. J Cell Biol. 129: 1103-14, 1995.

43. Huff, S. Y., Quilliam, L. A., Cox, A. D., and Der, C. J. R-Ras is regulated by activators and effectors distinct from those that control Ras function. Oncogene. 14: 133-43, 1997.

44. Au, W. C., Moore, P. A., Lowther, W., Juang, Y. T., and Pitha, P. M. Identification of a member of the interferon regulatory factor family that binds to the interferon-stimulated response element and activates expression of interferon-induced genes. Proc Natl Acad Sci U S A. 92: 11657-61, 1995.

45. Bellingham, J., Gregory-Evans, K., and Gregory-Evans, C. Y. Mapping of human interferon regulatory factor 3 (IRF3) to chromosome 19q13.3-13.4 by an intragenic polymorphic marker. Ann Hum Genet. 62: 231-4, 1998.

46. Scott, H. S., Antonarakis, S. E., Lalioti, M. D., Rossier, C., Silver, P. A., and Henry, M. F. Identification and characterization of two putative human arginine methyltransferases (HRMT1L1 and HRMT1L2). Genomics. 48: 330-40, 1998.

47. Abramovich, C., Yakobson, B., Chebath, J., and Revel, M. A protein-arginine methyltransferase binds to the intracytoplasmic domain of the IFNAR1 chain in the type I interferon receptor. Embo J. 16: 260-6, 1997.

48. Apte, S. S., Mattei, M. G., and Olsen, B. R. Mapping of the human BAX gene to chromosome 19q13.3-q13.4 and isolation of a novel alternatively spliced transcript, BAX delta. Genomics. 26: 592-4, 1995.

49. Oltvai, Z. N., Milliman, C. L., and Korsmeyer, S. J. Bcl-2 heterodimerizes in vivo with a conserved homolog, Bax, that accelerates programmed cell death. Cell. 74: 609-19, 1993.

50. Jackson, P. and Baltimore, D. N-terminal mutations activate the leukemogenic potential of the myristoylated form of c-abl. Embo J. 8: 449-56, 1989.

51. Hirai, H. and Varmus, H. E. Site-directed mutagenesis of the SH2- and SH3-coding domains of c-src produces varied phenotypes, including oncogenic activation of p60c-src. Mol Cell Biol. 10: 1307-18, 1990.

52. Yin, X. M., Oltvai, Z. N., and Korsmeyer, S. J. BH1 and BH2 domains of Bcl-2 are required for inhibition of apoptosis and heterodimerization with Bax [see comments]. Nature. 369. 321-3, 1994.

53. Chittenden, T., Harrington, E. A., O'Connor, R., Flemington, C., Lutz, R. J., Evan, G. I., and Guild, B. C. Induction of apoptosis by the Bcl-2 homologue Bak. Nature. 374: 733-6, 1995.

54. Simonian, P. L., Grillot, D. A., Andrews, D. W., Leber, B., and Nunez, G. Bax homodimerization is not required for Bax to accelerate chemotherapy-induced cell death. J Biol Chem. 271: 32073-7, 1996.

55. Ottilie, S., Diaz, J. L., Chang, J., Wilson, G., Tuffo, K. M., Weeks, S., McConnell, M., Wang, Y., Oltersdorf, T., and Fritz, L. C. Structural and functional complementation of an inactive Bcl-2 mutant by Bax truncation. J Biol Chem. 272: 16955-61, 1997.

56. Akahani, S., Nangia-Makker, P., Inohara, H., Kim, H. R., and Raz, A. Galectin-3: a novel antiapoptotic molecule with a functional BH1 (NWGR) domain of Bcl-2 family. Cancer Res. 57: 5272-6, 1997.

57. Gout, I., Dhand, R., Hiles, I. D., Fry, M. J., Panayotou, G., Das, P., Truong, O., Totty, N. F., Hsuan, J., Booker, G. W., and et al. The GTPase dynamin binds to and is activated by a subset of SH3 domains. Cell. 75. 25-36, 1993.

58. Pleiman, C. M., Hertz, W. M., and Cambier, J. C. Activation of phosphatidylinositol-3′ kinase by Src-family kinase SH3 binding to the p85 subunit. Science. 263: 1609-12, 1994.

TABLE 1
EST clones with >98% identity to exons of the BPR gene

| | | | Homologous |
|---|---|---|---|
| GenBank Accession # | | I.M.A.GE. ID | exons |
| AI434190 | Stomach | 2132102 | 1 |
| A251393 | Testis | 684243 | 1-4 |
| 76415 | Fetal Heart | 345104 | 1-4 |
| H72004 | Mammary | 214940 | 1-4 |
| AA6419448* | Testis | 1183976 | 3-7 |
| AA237057 | Testis | 683907 | 3-7 |
| R38463 | Placenta | 136942 | 5-6 |
| AA649065* | Testis | 1186334 | 6-7 |

*Contains a Poly(A) tail

TABLE 2
Primers used for reverse transcription polymerase
chain reaction (RT-PCR) analysis of the BPR and
Actin genes.

| | | | |
|---|---|---|---|
| Gene | Primer name | Sequence¹ | Length of PCR Product |

| | | | |
|---|---|---|---|
| BPR | BPR-F1 | TAA CAG ACC CAA AAG CCG ATG | |
| | BPR-R7 | AGT CCA CGG GTG AAA CAG CC | 1003 and 860 |
| | BPR-F2 | GGA GAC CGC AAG TTG AGT GG | |
| | BPR-R6 | GTC ATC CCG GCT ACA GAA CA | 556 and 413 |
| | BPR-R2 | CCT CAC CAC GCC TAA GGA AG | |
| | BPR-R3 | GGG AAG ACA TCT TCG AAG GC | |
| Actin | AGTINS | ATC TGG CAC CAC ACC TTC TA | |
| | AGTINAS | CAT ACT CCT GCT TGC TG | 838 |

¹All nucleotide sequence are given in the 5′→3′orientation.

TABLE 3
Putative post-translational modification sites in the novel BPR gene

| Modification | Residue | Position¹ |
|---|---|---|
| O-glycosylation | Thr | 117 |
| | Ser | 113, 121, 263, 273 |
| Protein Kinase C phosphorylation | Thr | 97, 204 |
| | Ser | 151, 259, 287 |
| Casein Kinase II phosphorylation | Thr | 204 |
| | Ser | 255, 325 |
| N-myristoylation | Gly | 82, 145, 292, 294, 295, |
| | | 316 |

¹The residues are numbered according to the sequence shown in FIG. 1 and GenBank submission (Accession # AF289220).

1. An isolated BPR nucleic acid molecule of at least 30 nucleotides which hybridizes to one of SEQ ID NOs. 1 to 8, or the complement of SEQ ID NO. 1 to 8, under stringent hybridization conditions.

2. An isolated nucleic acid molecule as claimed in claim 1 which comprises:
(i) a nucleic acid sequence encoding a protein having substantial sequence identity with the amino acid sequence shown in SEQ. ID. NO 24 or 25;
(ii) nucleic acid sequences complementary to (i);
(iii) a degenerate form of a nucleic acid sequence of (i);
(iv) a nucleic acid sequence comprising at least 18 nucleotides and capable of hybridizing to a nucleic acid sequence in (i), (ii), or (iii);
(v) a nucleic acid sequence encoding a truncation, an analog, an allelic or species variation of a protein comprising the amino acid sequence of SEQ. ID. NO.24 or 25; or
(vi) a fragment, or allelic or species variation of (i), (ii) or (iii).

3. An isolated nucleic acid molecule as claimed in claim 1 which comprises:
(a) a nucleic acid sequence having substantial sequence identity or sequence similarity with a nucleic acid sequence of one of SEQ. ID. NOs. 1 to 8;
(b) nucleic acid sequences complementary to (i), preferably complementary to the full nucleic acid sequence of one of SEQ. ID. NOs. 1 to 8;
(c) nucleic acid sequences differing from any of the nucleic acid sequences of (i) or (ii) in codon sequences due to the degeneracy of the genetic code; or
(d) a fragment, or allelic or species variation of (i), (ii) or (iii).

4. An isolated nucleic acid molecule as claimed in claim 1 which encodes a protein which binds an antibody of a BPR protein.

5. A regulatory sequence of an isolated nucleic acid molecule as claimed in claim 1 fused to a nucleic acid which encodes a heterologous protein.

6. A vector comprising a nucleic acid molecule of claim 1.

7. A host cell comprising a nucleic acid molecule of claim 1.

8. An isolated protein which contains a proline-rich domain and a BH2 domain.

9. An isolated protein as claimed in claim 8 comprising an amino acid sequence of SEQ. ID. NO. 24 or 25.

10. An isolated protein as claimed in claim 8 having at least 65% amino acid sequence identity to an amino acid sequence of SEQ. ID. NO. 24 or 25.

11. A method for preparing a protein as claimed in claim 9 comprising:
(a) transferring a vector as claimed in claim 6 into a host cell;
(b) selecting transformed host cells from untransformed host cells;
(a) culturing a selected transformed host cell under conditions which allow expression of the protein; and
(b) isolating the protein.

12. A protein prepared in accordance with the method of claim 11.

13. An antibody having specificity against an epitope of a protein as claimed in claim 9.

14. An antibody as claimed in claim 13 labeled with a detectable substance and used to detect the polypeptide in biological samples, tissues, and cells.

15. A probe comprising a sequence encoding a protein as claimed in claim 9, or a part thereof.

16. A method of diagnosing and monitoring a condition associated with a BPR by determining the presence of a nucleic acid molecule as claimed in claim 1.

17. A method of diagnosing and monitoring a condition associated with a BPR protein by determining the presence of a protein as claimed in claim 9.

18. A method for identifying a substance which associates with a protein as claimed in claim 9 comprising (a) reacting the protein with at least one substance which potentially can associate with the protein, under conditions which permit the association between the substance and protein, and (b) removing or detecting protein associated with the substance, wherein detection of associated protein and substance indicates the substance associates with the protein.

19. A method for evaluating a compound for its ability to modulate the biological activity of a protein as claimed in claim 9 comprising providing a known concentration of the protein with a substance which associates with the protein and a test compound under conditions which permit the formation of complexes between the substance and protein, and removing and/or detecting complexes.

20. A method for detecting a nucleic acid molecule encoding a BPR protein in a biological sample comprising the steps of:
(a) hybridizing a nucleic acid molecule of claim 2 to nucleic acids of the biological sample, thereby forming a hybridization complex; and
(b) detecting the hybridization complex wherein the presence of the hybridization complex correlates with the presence of a nucleic acid molecule encoding the protein in the biological sample.

21. A method for treating a condition mediated by a BPR protein comprising administering an effective amount of an antibody as claimed in claim 13.

22. A method for treating a condition mediated by a BPR protein comprising administering an effective amount of a compound identified in accordance with a method claimed in claim 21.

23. A composition comprising a protein as claimed in claim 9 and a pharmaceutically acceptable carrier, excipient or diluent.

24. Use of a protein as claimed in claim 9 in the preparation of a pharmaceutical composition for treating a condition mediated by the protein.

25. A transgenic non-human mammal which does not express or has reduced expression of a BPR protein as claimed in claim 9 resulting in a BPR associated pathology.


SEQUENCE LISTING (30 sequences)

SEQ ID NO: 1 - DNA, 9700 bp, Homo sapiens
ccggacccacctggcctggagtttccgcacccagacctccttctcacgggccacggcacaggtccttcacccatattttcgggggtacaaacaggaaacctcctcttcccatcctcccatcctcccgagaggctctctaatcagctagggttccttcccatatccatggcacatccttccccagcgtcttcagtgcagaccctccactccctcaattccgccagtatagacctcctccctcccccacatcatcgaaagtaagtatccccactttcagggtagcatcctctttccctagttatttcagtgcagaatcgattcacgtgtaagcctctacctcctgcacttaccccaatgtagacccccttccacagtccccctacagaagatctcccaaaacatttccaacacagaacttccattcaattcccctccgactgcccgcctccctcctccaggacagagcacgccccaggtttcgagactgaactacccccatctacggagtccacccctccctctctagaaccccctcagtgtagacgcctcctcttttcctctttcctccctactttttctagtttttctgcagccgacctccagcgtcggccactgtagctccttccttgctccactttccccagagtacacagcctgcctttccactctccgtgcagacccatcctctttcccgctcctcgctctcggacgccaccaacgctctcccgggctcttccgcgttacctacgatggaaggtcggggcgtgcgggcagctggaacccacccctgtcttggagctccgggtagctctcaaactcgaggctgcgcaccccctttcccgtcagctgagctctagagcgctggggctttctttttgatcgacttcttgaaataaaaccaactttatcattctttgggtaacagacccaaaagccgatgggacggcccgctgggctgttcccgcccctatgcccttttttgggtttccggccagaggcatgctgggagcgtcacatgcaaattgagcgtgcacccagcgttccgccctttctacgctgggccggttatcgacccggcccagtgcgcaggcgcgggaaagttgaactaataaagtttgtacgagttcagtggaggagaccgcaagttgagtggaggaggcggcggtggggccccggaccaggtcagcggggtgttgacgaggggtggggtgaggagggaagaggagggggccgggatccagtggtgggcaccccagtcccgcttctctgatatccaagggtccagggtcctctagattccagcaggggtcggagatttggaactacacctcacagcaggctgcgcggcgaagaagcgcggacctaggggaagatttgccctgaaacctcttgggattggtagttcccgatgctttaagggtttcaggagtttggcatatgcctcttatagaatccaggagttggggcgctaagcctcttcttcctgggagacctaagggtctggggctccagttcctttttctctaagactcagaagtccaggaccccagctccctcctcctttagacccacgaatctgggcacccagccccaccacctttccttggatcgaagagtctttttcctgatcgctttttcgtctgaggactcaagagtctaggcccccagacccacactctccaaaggacccaggagtccaggccctcagcaccttccttgggactcacaagtcctggtccccaagcctccacttcttgctaaacccttggagtccagtccctaaccctctctctcacaggtgcctccatggcaggctctgaagagctggggctccgggaagacacgctgagggtcctagctgccttccttaggcgtggtgaggctgccgggtctcctgttccaactccacctaggtaagaggagtggcccttctcccccaggggccagcatttgaggctccctcctaactcttgctcctggtctcagtctgtctcctctctttatatctggctctattctctgtctttggggttttggagagaggtcctcagcgggggagggagggagttggggaaagagactgcttgcaggggatctctggtggggtcagtctctgaagtcctcctctccagaagccctgcccaagaagagccaacagacttcctgagccgccttcgaagatgtcttccctgctccctggggcgaggagcagccccctctgagtcccctcggccttgctctctgcccatccgcccctgctatggtttagagcctggtaagagatttccatgatcatctatgaagccggcagaacacgggataaggtgctagtttaagatcactcagctagggggtggctgtgtggagtctctagcaccagccttcaggacagaaccgtcctgtcctctcccctccccagttagatttctgtcaattctgtgggttggctttcagagagcatttggtcaatctggttgttgtctctttccttccataactacttccctgcccctaggacttttgtagccaggaatacccctgatagaggggcagaagggctggttttcaatcacatctccctggctgttttagttgcgtgactctggggaaaatgctttctcttccctgctcccctgtttttccaatagcagagtaggaattggggaggaggtcactggacttgatcaaggtgacccacaagtttcctcataggtgtgaacctggacagactagtaatggctgctgggagtgctagcttgaaaaggggttcacagcttgggagaggaagtgctgtgatcaattggtgatgtctgtagtgagtgagggaaggggatactaacagtaatgtcttgtgtttgccttccttggatgagatcatcttcaaggtctagttgagctctgacagtcttttggagacagattgttgctctgtcacccaggctggagtgcaggggtgcaatctcgactcactgcaatccccacctcctgggttcaagcgattctcgtgcctcagcctcccgagtgactgggattacaggtgcacgccagcatactagctgatttttatacttttagtagacacggggtttcgccacgttggccaggctggcctcaaactcctggcctcaagtgaccctcccacgtcggcctcccaaaatgttgagattgcagacgtgagccaccgcaccctgtcagctctgacattcttttttttttttttttttttgagacggagtttcgcccttgttgcccaggctggagtgcaatggcccgatctcggctcaccacaacctctacctcccgggttcaagtaattctcctgcctcagcctcccaagtagctgggattataggcatgcgccaccatgcccggctaattttttttgtatttttagtagagatggggtttctccatgttggtcaggctgctctcgaactcccaacctcaggtgatctgcctgcctcagcctcccaaagtgatgggattacaggcgtgagccactgcgcctggccccagctctgacattctaagtccagtctgaggatctcataaagtgaaaagtacaagggttttggcatttttaaccaagttttctcactgggcctcagtttaccccattataaaatgggaatcatggccaggcttggttgctcacgcctgtaatcctagcactttgggaggctgaggtgggtggattgcctgaggtcagcctgggcaacacggtgaaaccccatttctactaaaatacaaaaaattagccaggtatggcggcgtgcgcctgtagtcccagctactcatgaggctgaggcaggagaattgcgagactccgtttcccaaaatcaatacataaataaataataaataaaatgggaatcattgccacctgccaaggtagttgggggaaggagagaatccgtaagtttccaatgtcctggaaacctgtcattaactcttttcaccccaggcccagctactccagacttctatgctttggtggcccagcggctggaacagctggtccaagagcagctgaaatctccgcccagcccaggtgaggcacagaggagccccagagaaggatggcggtgggaggatagtcagggttctgcccccagccaaattctcttctgcctccagaattacagggtcccccatcgacagagaaggaagccatactgcggaggctggtggccctgctggaggaggaggcagaagtcattaaccagaaggtgatgggcatctgtcccactccttggcaaggacaggagttgcggaatggtggggcactgggatcagaagctggatctgtattttctttggatggtcaggtggaaagaggctgggacaaggtttcaatgaagggttgaggccgggtgtggtggctcacgcctgtaatcccagcactttgggaggccgagttgggtggatgacttgaggtcaggagttcgagaccagcctggccaacatggtgaaaccccatctctactaaaaatacaaaaatcggacaggtgtggtggtgcgcttctgtaatcccagctactcagaaggctgaggcaggagaatcgcttgagcccaggaggtggaggttgcagtgagccaagattgcgccactgcactccagcctgggtgacagagtgagactctgtctttaaaaaaaaaaaaaaaaaagaatagttgaatgacaggggtatggttagggtagtactgtaccgtaaattagttgttttaattgattgtttagaggggcatggcctaaatgcagagggtgtggccatcagaaacattatccacaagagactggccaagctggggaagtaagtgaatgaatgtgggggtggggggagtggctaaaatcttactgagcatagaagggacctggcttagaatgtaggggaaaatgtactaccagaggaggtgtggcttgtggctgtgtcctggaatctgattggcttcggagcttggacaaggtacatcgtcctttaatattaattgaatggtagagggcaagattgttgtagacactaggttggcccctgggaaaggtcaccaggggcatggctagattaactcagttacagtagagattggccctgattggctgttgaggcaagggcctggcatggcttagggactcacgttccaggccactatcccgggggtagtttggcagagaccctggagtctggcttgattggctaatatggatgaggggtggcctagaaccttagcatctagaacgatcctgaaccgggaagccacgcctcccggccctctattggctggccccgggggcgctgctgacgcggaccctgcctcttccaccctacagctggcctcggaccccgccctgcgcagcaagctggtccgcctgtcctccgactctttcgcccgcctggtggagctgttctgtagccgggatgacagctctcgcccaagccgagcatgccccgggcccccgcctccttccccggagcccctggcccgcctggccctagccatggagctgagccggcgcgtggccgggctggggggcaccctggccggactcagcgtggagcacgtgcacagcttcacgccctggatccaggcccacgggggctgggtgagccgctgaagcctctctctccgggcctcactttacctaactgtcatgtgatagaggaggggcggggacttcttaggttcctctcagacctcaggtattctcccagctccactgcgttcgttctgttgagcccttgctgtatgccaggactgcagcgatgagtgcagacactatgcctggcgtcaacgactgggtacgctaatactgtgagcgtgaaaccagtgactgaggttactcagggagaggccggacggggtagctcatgcctgtaatcccagcacttttggaggccaaggcaggagaattatttgaggccaggagttcaagaccagcctggacaacatagtaagaccccatctctacaaaaattgaaaataactagccagggtcgggtgcggtggctcacacctgtaatcccagcactttgggaggccgaggcaggtggatcacctgaggtcaggagtttgagaccagctggccaacgtggtgaaaccctgtgtctactaaaaaaaatacataaattagccgggcgcggtggcaggtgcctgtaatcccagctaccgaggagactgaggcatgagaatcgcttgaacctgggaggcagagattgcagtgagctgagatcatgccactgcactccagcctgggcgacagagtgagactgtgtctcaacaaaataatataataaaaacctggtgcggtggctcacacctgtaatcccagcactttgggagactgaggtgggtggatcacttgaggtcaggagtttgagaccagcctgaccatggtgacattcagtctctactaaaaatacaaaattagcccggcatgcctgtaatcccagctagggaggctgaggcaagagaattgcttgaacctgggaggcagaggttgtagtgagccgagattgcaccactgcactccagcctgggcaacaagagcggggctctgtctcaaaaaataaataaatacaaatttttaaaattaataataattagcaaagcatggtagctcacacgtttactcccagctactcaggaggcagaggagccgggaggatcacttgagcccaggagttcaaggctgtggtgagctatgattgcgccactgtactccagtgtgggtgacagagtgggaccctgtctcaaaaaataaaatagtgagagtagtagtgctacctcaaagggtcgctctgagggttaaaatggggtcaatgtggtaagccctctgtgcactgcctggcatgcaggcattttgttggtgaggttatatggctacctgctgcctttccaacagtgcctgccaggatgccctcccggggaaccccctgcctccgtcctgctccttctggtgggctctggtttcacccccaacgaggccttcccaggtccccattgacaatcacagccacctgcacaccctcccaccgccctccttcccccttcctgctttacttttccttccatagcactttgggccttccaacagatggtggaattgacttgttttacttgtcttctggtttatttttctttgttggtgtctcccacctctaacacatcagcttcaggagacccagaagtcttcccctctccttttcttactttatctccagtgccggtttcagagttggattgggctccagcgtgctggccctgtggcttggcgactgtcgcagtctccctgagcggcactctccccactggtccagtgagcaccgtctcatttacctgccttggggggtcgctgtgggtattcactgagagagggacacaagccaggctctggatggaaccggtttccctggcaggagctggtcaagtgtgtttgaggagcagggaggaggaggccagtagggctacagtagggtgagcgggcgacagatgagaggaggagaggcgccgggccagccaggaccctgtggtcacggtgagtgacaactcggactttgactctgggtaagccaaggtctctgagcagcggagggacaggcgctgatttaggcgctaatgggcgccctctggcggctgcctggggacagactgctgggggcgagggcagaagtaggggcacagggaggaggtgagtggatctcacccaagtcgggggcggtggagaatgctggatttaacttagcggtgaagctgacagatttggctaatgggccagatgtggggacagactaaaggatgctggcccgagcagctgcagggcgacatcttaagttccccgagatggggaaggtgtggggatgggggcgatgggggtggggagcaggactggaggtggtccggtctacagtaagatcagaactcatagcacatgttgaggggaatggatataagtgttgggctcagcaaagaagctcgtttattttattttattttaatttttcagatggagtctcactctattgcccagactggagtgcagtggcacgaccttggctcactgcaacctccacctcctgggttcaagcgattccctgcctcagcctcctgagtagctggggttacaggcgcacaccaccatgtccggctaattttgtagttttagcagagacggggttttgccctgttggccaggctggtctcgaactcttggcctcaagtgatccacccacctcggcctcccaaagtgctggaattacaggctgagctaccacgcctggacaatttcttaaaaaatttttttaaagtaggattgatagcagatgagttttctcggtgggccaggtcctctgctagggatttcaatggaatgatcccatttaaacccactacaacctcatggggctcttattgtcctcgtcttgtgggggcagagaggaaaagtccccagcatgcagagtgttggaatctggatttgtacacaaagccacgactctgaattgttcaaatcccaagactctcttccccatctctgactctgtcccccaactcttcactctctctggatctctgtctaatcccactccagtgtcctgaccctcctcgagctcaacctttcctcctcctgctaggaccctgccccctcatccctgtgcccttctgtctgggtctctgtccccttgtctgggtgtctgtcaccctccgctctctggttcctcgccttcccgtctctaatctcacagggctgatgtggacgctgctgtatggggggaacacctgccctgctcacagggctctgccacttttcccttccaggagggcatcctggctgtttcacccgtggacttgaacttgccattggactgagctctttctcagaagctgctacaagatgacacctcatgtccctgccctcttcgtgtgcttttccaagtcttcctattccactcagggctgtggggtggtggttgccctacctgtttttgccaaaaataaattgtttaaaacttttcttattaaaaacgttacaaagtattcaattgtctgttcttacacttgggaactaaagttttaaggagaggagatttggttcagagatttctgtggcttacgcaaattgaggaaagaaccagttcattcattctagaaatatttatttgcagcacctacaagagcctgtctcctggacatgtaacatatttgagagaatacctgtaaatattccagcaacttgcatttttttttcctattgcacgatctggttattctagctaatgctgtatgatggtcatggagtctttccagtgaattccacagaaaggtacagtctaacttcacctgtgtctaggacaccaccctcttcttgtttttctcctcccattgctcctttcaagtctctcttgctggtttctcatctacctacagaccagtggtttgtctcacacacaccccccactctccctttttttttggagatagggtcttgctctgttgcctatacttgcactgttatatagatacagtggcactatcatggctcactgcagcctcaacctcctgggctcaagtgatcctcccacttcagcctctctagtagctggaactaaaggcatgcgacacgacgcctagctaatttttaaaaaagaatttttatgggaggccaaggcaggtggatcacttgaggtcaggagttcaagaccagcca

SEQ ID NO: 2 - DNA, 926 bp, Homo sapiens
aagtaagtatccccactttcagggtagcatcctctttccctagttatttcagtgcagaatcgattcacgtgtaagcctctacctcctgcacttaccccaatgtagacccccttccacagtccccctacagaagatctcccaaaacatttccaacacagaacttccattcaattcccctccgactgcccgcctccctcctccaggacagagcacgccccaggtttcgagactgaactacccccatctacggagtccacccctccctctctagaaccccctcagtgtagacgcctcctcttttcctctttcctccctactttttctagtttttctgcagccgacctccagcgtcggccactgtagctccttccttgctccactttccccagagtacacagcctgcctttccactctccgtgcagacccatcctctttcccgctcctcgctctcggacgccaccaacgctctcccgggctcttccgcgttacctacgatggaaggtcggggcgtgcgggcagctggaacccacccctgtcttggagctccgggtagctctcaaactcgaggctgcgcaccccctttcccgtcagctgagctctagagcgctggggctttctttttgatcgacttcttgaaataaaaccaactttatcattctttgggtaacagacccaaaagccgatgggacggcccgctgggctgttcccgcccctatgcccttttttgggtttccggccagaggcatgctgggagcgtcacatgcaaattgagcgtgcacccagcgttccgccctttctacgctgggccggttatcgacccggcccagtgcgcaggcgcgggaaagttgaactaataaagtttgtacgagttcagtggaggagaccgcaagttgagtggaggaggcggcggtggggccccggaccag

SEQ ID NO: 3 - DNA, 115 bp, Homo sapiens
gtgcctccatggcaggctctgaagagctggggctccgggaagacacgctgagggtcctagctgccttccttaggcgtggtgaggctgccgggtctcctgttccaactccacctag

SEQ ID NO: 4 - DNA, 143 bp, Homo sapiens
aagccctgcccaagaagagccaacagacttcctgagccgccttcgaagatgtcttccctgctccctggggcgaggagcagccccctctgagtcccctcggccttgctctctgcccatccgcccctgctatggtttagagcctg

SEQ ID NO: 5 - DNA, 87 bp, Homo sapiens
gcccagctactccagacttctatgctttggtggcccagcggctggaacagctggtccaagagcagctgaaatctccgcccagcccag

SEQ ID NO: 6 - DNA, 92 bp, Homo sapiens
aattacagggtcccccatcgacagagaaggaagccatactgcggaggctggtggccctgctggaggaggaggcagaagtcattaaccagaag

SEQ ID NO: 7 - DNA, 273 bp, Homo sapiens
ctggcctcggaccccgccctgcgcagcaagctggtccgcctgtcctccgactctttcgcccgcctggtggagctgttctgtagccgggatgacagctctcgcccaagccgagcatgccccgggcccccgcctccttccccggagcccctggcccgcctggccctagccatggagctgagccggcgcgtggccgggctggggggcaccctggccggactcagcgtggagcacgtgcacagcttcacgccctggatccaggcccacgggggctgg

SEQ ID NO: 8 - DNA, 216 bp, Homo sapiens
gagggcatcctggctgtttcacccgtggacttgaacttgccattggactgagctctttctcagaagctgctacaagatgacacctcatgtccctgccctcttcgtgtgcttttccaagtcttcctattccactcagggctgtggggtggtggttgccctacctgtttttgccaaaaataaattgtttaaaacttttcttattaaaaacgttacaaa

SEQ ID NO: 9 - DNA, 21 bp, Artificial Sequence
  Segments
taacagacccaaaagccgatg

SEQ ID NO: 10 - DNA, 20 bp, Artificial Sequence
  Segments
agtccacgggtgaaacagcc

SEQ ID NO: 11 - DNA, 20 bp, Artificial Sequence
  Segments
ggagaccgcaagttgagtgg

SEQ ID NO: 12 - DNA, 20 bp, Artificial Sequence
  Segments
gtcatcccggctacagaaca

SEQ ID NO: 13 - DNA, 20 bp, Artificial Sequence
  Segments
cctcaccacgcctaaggaag

SEQ ID NO: 14 - DNA, 20 bp, Artificial Sequence
  Segments
gggaagacatcttcgaaggc

SEQ ID NO: 15 - DNA, 20 bp, Artificial Sequence
  Segments
atctggcaccacaccttcta

SEQ ID NO: 16 - DNA, 17 bp, Artificial Sequence
  Segments
catactcctgcttgctg

SEQ ID NO: 17 - protein, 16 aa, Artificial Sequence
  Segments
WIQAHGGWEGILAVSP

SEQ ID NO: 18 - protein, 16 aa, Artificial Sequence
  Segments
WIQDNGGWDAFVELYG

SEQ ID NO: 19 - protein, 16 aa, Artificial Sequence
  Segments
WIQDQGGWDGLLSYFG

SEQ ID NO: 20 - protein, 16 aa, Artificial Sequence
  Segments
WIHSSGGWAEFTALYG

SEQ ID NO: 21 - protein, 16 aa, Artificial Sequence
  Segments
WIQENGGWDTFVELYG

SEQ ID NO: 22 - protein, 16 aa, Artificial Sequence
  Segments
WIAQRGGWVAALNLGN

SEQ ID NO: 23 - protein, 16 aa, Artificial Sequence
  Segments
WLRRRGGWTDVLKCVV

SEQ ID NO: 24 - protein, 334 aa, Homo sapiens
MGRPAGLFPPLCPFLGFRPEACWERHMQIERAPSVPPFLRWAGYRPGPVRRRGKVELIKFVRVQWRRPQVEWRRRRWGPGPGASMAGSEELGLREDTLRVLAAFLRRGEAAGSPVPTPPRSPAQEEPTDFLSRLRRCLPCSLGRGAAPSESPRPCSLPIRPCYGLEPGPATPDFYALVAQRLEQLVQEQLKSPPSPELQGPPSTEKEAILRRLVALLEEEAEVINQKLASDPALRSKLVRLSSDSFARLVELFCSRDDSSRPSRACPGPPPPSPEPLARLALAMELSRRVAGLGGTLAGLSVEHVHSFTPWIQAHGGWEGILAVSPVDLNLPLD

SEQ ID NO: 25 - protein, 176 aa, Homo sapiens
MGRPAGLFPPLCPFLGFRPEACWERHMQIERAPSVPPFLRWAGYRPGPVRRRGKVELIKFVRVQWRRPQVEWRRRRWGPGPGASMAGSEELGLREDTLRVLAAFLRRGEAAGSPVPTPPRPSYSRLLCFGGPAAGTAGPRAAEISAQPRITGSPIDREGSHTAEAGGPAGGGGRSH

SEQ ID NO: 26 - protein, 8 aa, Artificial Sequence
  Segments
WIQXXGGW

SEQ ID NO: 27 - protein, 8 aa, Artificial Sequence
  Segments
EGILAVSP

SEQ ID NO: 28 - DNA, 11 bp, Artificial Sequence
  Segments
gccagccatgg

SEQ ID NO: 29 - DNA, 6 bp, Artificial Sequence
  Segments
aataaa

SEQ ID NO: 30 - DNA, 6 bp, Artificial Sequence
  Segments
attaaa